(19) United States
(12) Patent Application Publication
(10) Pub. No.: US 2025/0206786 A1
(43) Pub. Date: Jun. 26, 2025

(54) CRY PROTEIN CRYSTALS AND METHODS OF USE

(71) Applicant: Battelle Memorial Institute, Columbus, OH (US)

(72) Inventors: Bradley Heater, Columbus, OH (US); Jonathon Clifton, Columbus, OH (US); Anurup Krishna, Columbus, OH (US); Katarzyna Kucharzyk, Columbus, OH (US); Steven Risser, Columbus, OH (US)

(21) Appl. No.: 18/649,688

(22) Filed: Apr. 29, 2024

Related U.S. Application Data
(60) Provisional application No. 63/613,569, filed on Dec. 21, 2023.

(51) Int. Cl. C07K 14/325; A01N 63/50; A01P 1/00; A01P 3/00; C12N 15/113

(52) U.S. Cl. CPC C07K 14/325; A01N 63/50; A01P 1/00; A01P 3/00; C12N 15/113; C12N 2310/14

(57) ABSTRACT

Provided are Cry3Aa protein crystals comprising a negatively-charged biomolecule encapsulated therein. The solvent channel of the Cry3Aa protein crystal comprises one or more amino acid substitutions of an uncharged or negatively charged amino acid for a positively charged amino acid. Encapsulation of the biomolecule comprises interaction of the biomolecule with the one or more positively charged amino acids in the solvent channel. Negatively-charged biomolecules of interest include nucleic acids and negatively-charged proteins. In some instances, the negatively-charged biomolecule is an RNA (e.g., an siRNA) adapted to reduce expression of a target gene by RNA interference (RNAi). Methods of using the Cry3Aa protein crystals are also provided. For example, provided are methods of controlling a pest, a bacterium, a virus, a fungus, or a parasite using the Cry3Aa protein crystals. Methods of producing the Cry3Aa protein crystals are also provided.

### CROSS-REFERENCE TO RELATED APPLICATIONS

This application claims the benefit of U.S. Provisional Patent Application No. 63/613,569, filed Dec. 21, 2023, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, “BAMI-001_SEQLIST.xml”, created on Apr. 26, 2024 and having a size of 28,155 bytes. The contents of the Sequence Listing XML are incorporated herein by reference in their entirety.

### INTRODUCTION

The global insecticide market was nearly 17 billion U.S. dollars in 2020, and it is expected to climb to nearly 23 billion dollars by 2025. The insecticide market is dominated by small chemical compounds, but owing to potential toxicity to non-invasive species, increased insect resistance, damage to the environment, and unwanted problems associated with human health, industries are pursuing alternative solutions such as bioinsecticides. Bioinsecticides are derived from natural materials, such as RNA and proteins. They are biodegradable and highly selective. Consequently, bioinsecticides are gaining headway in the insecticide market, with a value of 2.2 billion U.S. dollars in 2020, which is expected to double to 4.6 billion by 2025.

In addition to the importance of biologicals in the agricultural industry, there is recent interest in developing platforms to protect food sources as a national security goal. For instance, release of an invasive species could destroy large areas of crops and reduce the food supply. Biological pesticides that would be beneficial in this circumstance include nucleic acid-based molecules targeting very specific genes (e.g., using the RNA interference (RNAi) pathway), yet their sequence can be tailored to affect a wide variety of invasive species. Unfortunately, such nucleic acid-based molecules (e.g., siRNA molecules) are unstable in the environment, so they are not effective as sprays for foliar applications.

### SUMMARY

Provided are Cry3Aa protein crystals comprising a negatively-charged biomolecule encapsulated therein. The solvent channel of the Cry3Aa protein crystal comprises one or more amino acid substitutions of an uncharged or negatively charged amino acid for a positively charged amino acid. Encapsulation of the biomolecule comprises interaction of the biomolecule with the one or more positively charged amino acids. Negatively-charged biomolecules of interest include nucleic acids and negatively-charged proteins. In some instances, the negatively-charged biomolecule is an RNA (e.g., a short interfering RNA (siRNA)) adapted to reduce expression of a target gene by RNA interference (RNAi). Methods of using the Cry3Aa protein crystals are also provided. For example, provided are methods of controlling a pest, a bacterium, a virus, a fungus, or a parasite using the Cry3Aa protein crystals. Methods of producing the Cry3Aa protein crystals are also provided.

### BRIEF DESCRIPTION OF THE FIGURES

FIG. 1: Illustration showing a mode of siRNA targeting specific pests while leaving others unharmed.

FIG. 2: Schematic illustration of an siRNA encapsulation approach. Bacillus thuringiensis (Bt) cells produce a native protein called Cry3Aa, which forms crystal inside the cell. These crystals are comprised of ˜5 nm×˜5 nm solvent channels, which are large enough to accommodate negatively-charged biomolecules, e.g., nucleic acids such as siRNAs, etc.

FIG. 3A-3C: Production and characterization of first generation Cry3Aa variant for siRNA encapsulation. (a) SDS-PAGE analysis of Cry3Aa wild-type and SupPos3A crystals. (b) Optimization of parameters for siRNA encapsulation into SupPos3A (for each ratio, bars from left to right are: 2 hours, 4 hours and 8 hours) and (c) encapsulation comparison of Cry3Aa and SupPos3A using optimized parameters. For each time point, bars from left to right are: Cry3Aa and SupPos3A.

FIG. 4A-4B: Approaches for encapsulation (a) Direct encapsulation approach where the crystals extracted from Bt are mixed directly with the siRNA. (b) Recrystallization approach where the crystals extracted from Bt are solubilized, mixed with the siRNA, and a pH change triggers recrystallization.

FIG. 5A-5C: Development of fluorescence-based assay for encapsulation. (a) Agarose gel after synthesis of FAM-labeled siRNA. (b) Encapsulation of FAM-siRNA by SupPos3A at two crystal concentrations. For each condition, bars from left to right are: 1 μM and 5 μM. (c) Fluorescence microscopy of Cry3Aa and SupPos3A crystals following direct encapsulation of fluorescein amidite (FAM)-siRNA.

FIG. 6A-6C: (a) SDS-PAGE analysis of panel of crystal mutants. The gel was overloaded, but an intense band was observed at the correct molecular weight for all constructs (b) Small scale solubilization of panel of crystal mutants at pH 10 and pH 11. For each condition, bars from left to right are: pH 10 and pH 11. (c) 15 mL scale solubilization of panel of crystal mutants at pH 11.

FIG. 7A-7B: Characterization of different crystal variants (a) Crystal fluorescence depicted as relative fluorescence units (RFU) following direct encapsulation of FAM-siRNA and washing. (b) Small scale recrystallization of the varying crystal mutants in the absence of siRNA, and subsequent resolubilization of the recrystallized crystals. For each condition, bars from left to right are: Recrystallized and Resolubilized.

FIG. 8A-8B: SEM analysis of Cry3Aa and Pos11R crystals generated in Bt. (a) SEM image of Cry3Aa wild-type crystals and (b) Pos11R crystals. Scale bar is 1 μm.

FIG. 9A-9B: Analysis of Pos11R S145C mutant. (a) Model showing the proximity of serine 145 residues at the crystallization interface in the Cry3Aa crystal structure. Mutation of serine 145 to cysteine would result in disulfide bond formation if the crystal lattice structure was retained (b) Small scale solubilization of Cry3Aa, Pos11R, and Pos11R at pH 11 in the presence or absence of DTT. Only Pos11R S145C requires DTT for solubilization, suggesting it forms the same crystal lattice structure as wild-type Cry3Aa. For each condition, bars from left to right are: −DTT and +DTT.

FIG. 10A-10D: Optimization of encapsulation by recrystallization in the presence of FAM-siRNA. (a) Encapsulation efficiencies of FAM-siRNA into Pos11R by recrystallization using varying Crystal:siRNA molar ratios (for each condition, bars from left to right are: Cry3Aa and Pos 11R), (b) after altering Pos11R protein concentration and buffer ratios, (c) and final optimization of buffer ratios. For each condition, bars from left to right are: 1:1 and 3:2. (d) Release of FAM-siRNA at different aqueous conditions. Large amounts of FAM-siRNA were released at pH 10. For each condition, bars from left to right are: 4:1 and 4.25:0.75.

FIG. 11A-11B: Increasing the siRNA loading into the crystal by altering the Pos11R: siRNA molar ratios. (a) Encapsulation efficiencies of FAM-siRNA into Pos11R at increasing concentration of siRNA and (b) % loading at increasing concentrations of siRNA.

FIG. 12A-12B: Encapsulation of larger siRNA molecules into Pos11R crystals (a) Validation that the FAM label on the siRNA was not the cause of the high loadings. (b) Encapsulation efficiencies of 60 bp, 80 bp, and 100 bp siRNA molecules after recrystallization of Pos11R. For each condition, bars from left to right are: Flour method and Agarose method.

FIG. 13: Comparison of Cry3Aa and Pos11R after encapsulation of FAM-siRNA by recrystallization. For each condition, bars from left to right are: Cry3Aa and Pos11R.

FIG. 14: Fluorescence microscopy of Cry3Aa wild-type and Pos11R crystals following encapsulation by recrystallization at different crystal protein:siRNA ratios.

FIG. 15: Fluorescence microscopy of smaller particles formed after recrystallization.

FIG. 16A-16B: Dynamic light scattering (DLS) data of Pos11R crystals post-recrystallization at different Pos11R:siRNA ratios. (a) DLS data depicting particle number distribution and (b) DLS data depicting volume distribution.

FIG. 17: Optimization of siRNA release from Pos11R crystals for screening stability. After direct binding, crystals were incubated at pH 10, pH 11, or in 0.1%, 0.5% and 1% SDS, and subsequently ran on an agarose gel and stained with ethidium bromide.

FIG. 18A-18C:RNAse protection assay of Pos11R-encapulated siRNA via recrystallization. (a) Schematic demonstrating theory of RNAse protection assay-siRNA present in solvent channels should be protected from RNAse-mediated degradation. (b) Agarose gel analysis of naked and encapsulated siRNA in the presence of increasing concentrations of RNAse. (c) % prevalence of siRNA left using densitometry analysis of the bands.

FIG. 19: Stability of siRNA in water and buffer over 24 hours. Naked and encapsulated siRNA were incubated in buffer or tap water. Samples were released from the crystal and loaded on the gel at different time points.

FIG. 20A-20B: Two-week stability of siRNA in tap water. (a) Agarose gel of naked and encapsulated siRNA at different time points over the two-week period and (b) densitometry analysis of band intensities to quantitate fraction siRNA remaining in the sample. For each condition, bars from left to right are: Day 3 and Day 14.

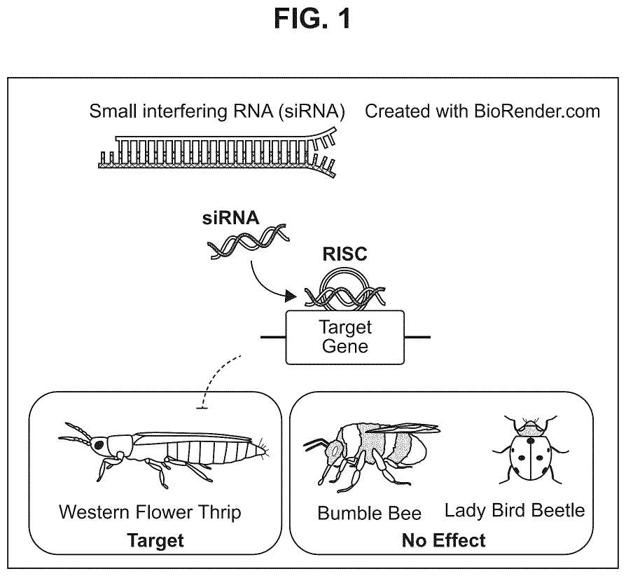

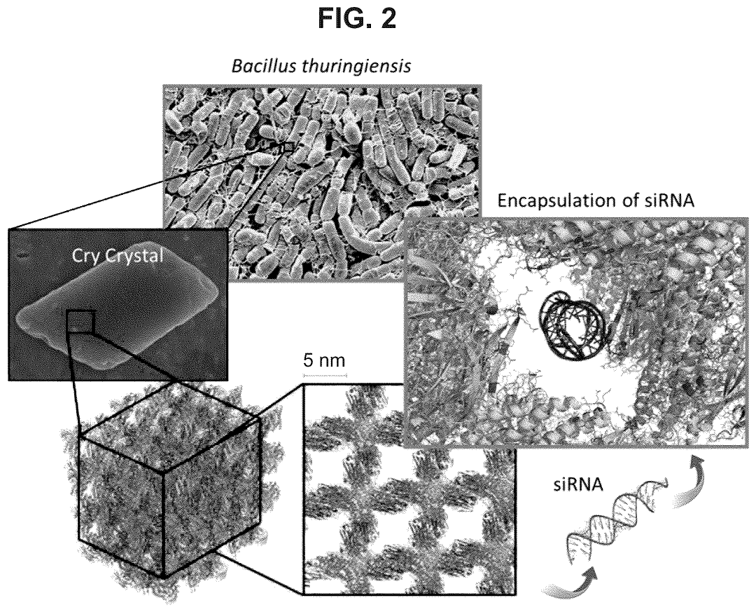

### DETAILED DESCRIPTION

Before the Cry3Aa protein crystals and methods of the present disclosure are described in greater detail, it is to be understood that the Cry3Aa protein crystals are not limited to particular embodiments described, as such may, of course, vary. It is also to be understood that the terminology used herein is for the purpose of describing particular embodiments only, and is not intended to be limiting, since the scope of the Cry3Aa protein crystals will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the Cry3Aa protein crystals. The upper and lower limits of these smaller ranges may independently be included in the smaller ranges and are also encompassed within the Cry3Aa protein crystals, subject to any specifically excluded limit in the stated range. Where the stated range includes one or both of the limits, ranges excluding either or both of those included limits are also included in the Cry3Aa protein crystals.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the Cry3Aa protein crystals belong. Although any Cry3Aa protein crystals similar or equivalent to those described herein can also be used in the practice or testing of the Cry3Aa protein crystals, representative illustrative Cry3Aa protein crystals are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present Cry3Aa protein crystals are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the Cry3Aa protein crystals, which are, for clarity, described in the context of separate embodiments, may also be provided in combination in a single embodiment. Conversely, various features of the Cry3Aa protein crystals, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present Cry3Aa protein crystals and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Cry3Aa Protein Crystals

Aspects of the present disclosure include Cry3Aa protein crystals comprising a negatively-charged biomolecule encapsulated therein. The solvent channel of the Cry3Aa protein crystal comprises one or more amino acid substitutions of an uncharged or negatively charged amino acid for a positively charged amino acid. Encapsulation of the biomolecule comprises interaction of the biomolecule with the one or more positively charged amino acids.

The Cry3Aa protein crystals of the present disclosure are based in part on the inventors' unpredictable findings that Cry3Aa protein crystals may be engineered to increase the positive charge of their solvent channels to enable encapsulation of negatively-charged biomolecules therein at high encapsulation efficiencies while retaining sufficient solubility of the crystals for release of the negatively-charged biomolecules at a target site of interest. For example, the engineered Cry3Aa protein crystals may be employed as biopesticides in which the solubility properties of the crystals provide for release of the negatively-charged biomolecule (e.g., nucleic acid such as an siRNA or the like, or negatively-charged protein) in the midgut environment of a pest. Moreover, the Cry3Aa protein crystals of the present disclosure provide an unpredictably high level of protection of the negatively-charged biomolecules from environmental conditions (e.g., enzymatic degradation, UV light, etc.) and long-term stability (e.g., in tap water), further supporting the use of the crystals for delivery of negatively-charged biomolecules in a wide variety of contexts including but not limited to biopesticide applications. Details of embodiments of the Cry3Aa protein crystals of the present disclosure will now be provided.

The terms “protein”, “polypeptide” or “peptide” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof.

As summarized above, the solvent channel of the Cry3Aa protein crystal comprises one or more amino acid substitutions of an uncharged or negatively charged amino acid for a positively charged amino acid. In certain embodiments, the one or more amino acid substitutions comprise substitution of an uncharged or negatively charged amino acid for an arginine (R) or lysine (K), e.g., an arginine. In some instances, the uncharged or negatively charged amino acid is an aspartic acid (D), glutamic acid (E), serine(S), asparagine (N), or glutamine (Q). According to some embodiments, the solvent channel of the Cry3Aa protein crystal comprises two or more of the amino acid substitutions, where the two or more substitutions are independently selected from a substitution of an aspartic acid (D), glutamic acid (E), serine(S), asparagine (N), or glutamine (Q) with an arginine (R) or lysine (K).

In some instances, a Cry3Aa protein crystal of the present disclosure comprises Cry3Aa proteins comprising an amino acid substitution selected from the group consisting of: D118R, S187R, S257R, S322R, N326R, N391R, N395R, E423R, Q430R, D432R, E433R, E466R, A629R, or any combination thereof, and wherein numbering is as in SEQ ID NO: 1. According to some embodiments, the Cry3Aa proteins from 2 to 13 of the substitutions, e.g., from 5 to 11 of the substitutions. For example, the Cry3Aa proteins may comprise the amino acid substitutions D118R, S187R, S257R, N326R and A629R. Such Cry3Aa proteins may further comprise the amino acid substitution N391R, E433R, E466R or any combination thereof, e.g., N391R, E433R and E466R. Such Cry3Aa proteins may further comprise the amino acid substitution E423R, Q430R, T436R or any combination thereof, e.g., E423R, Q430R and T436R.

According to some embodiments, the Cry3Aa proteins comprise one or more additional amino acid substitutions which do not involve substitution for a positively-charged amino acid but remove a negative charge while maintaining a similar amino acid structure. Non-limiting examples of such substitutions include E423Q, D432N, E433Q, E466Q, or any combination thereof. For example, the Cry3Aa proteins may comprise a D432N substitution.

Particular non-limiting examples of combinations of substitutions which may be present in the Cry3Aa proteins of the Cry3Aa protein crystals of the present disclosure include: D118R, S187R, S257R, N326R, E423Q, D432N, E433Q, E466Q, A629R (the combination of mutations of Cyr3Aa protein crystals sometimes referred to herein as “Pos5R”); D118R, S187R, S257R, N326R, N391R, E423Q, D432N, E433R, E466R, A629R (the combination of mutations of Cyr3Aa protein crystals sometimes referred to herein as “Pos8R”); and D118R, S187R, S257R, N326R, N391R, E423R, Q430R, D432N, E433R, T436R, E466R, A629R (the combination of mutations of Cyr3Aa protein crystals sometimes referred to herein as “Pos11R”).

The amino acid sequences of the wild-type Cry3Aa protein and non-limiting variants thereof of the present disclosure, and exemplary nucleotide sequences encoding the same, are provided in Table 1 below.

TABLE 1
Cry3Aa amino acid and nucleotide sequences

| Construct | Sequence | SEQ ID NO. |
|---|---|---|
| Cry3Aa wild-type | MNPNNRSEHDTIKTTENNEVPTNHVQYPLAETPNPTLEDL | 1 |
| amino acid | NYKEFLRMTADNNTEALDSSTTKDVIQKGISVVGDLLGVV | |
| | GFPFGGALVSFYTNFLNTIWPSEDPWKAFMEQVEALMDQK | |
| | IADYAKNKALAELQGLQNNVEDYVSALSSWQKNPVSSRNP | |
| | HSQGRIRELFSQAESHFRNSMPSFAISGYEVLFLTTYAQA | |
| | ANTHLFLLKDAQIYGEEWGYEKEDIAEFYKRQLKLTQEYT | |
| | DHCVKWYNVGLDKLRGSSYESWVNFNRYRREMTLTVLDLI | |
| | ALFPLYDVRLYPKEVKTELTRDVLTDPIVGVNNLRGYGTT | |
| | FSNIENYIRKPHLFDYLHRIQFHTRFQPGYYGNDSFNYWS | |
| | GNYVSTRPSIGSNDIITSPFYGNKSSEPVQNLEFNGEKVY | |
| | RAVANTNLAVWPSAVYSGVTKVEFSQYNDQTDEASTQTYD | |
| | SKRNVGAVSWDSIDQLPPETTDEPLEKGYSHQLNYVMCFL | |
| | MQGSRGTIPVLTWTHKSVDFFNMIDSKKITQLPLVKAYKL | |
| | QSGASVVAGPRFTGGDIIQCTENGSAATIYVTPDVSYSQK | |
| | YRARIHYASTSQITFTLSLDGAPFNQYYFDKTINKGDTLT | |
| | YNSFNLASFSTPFELSGNNLQIGVTGLSAGDKVYIDKIEF | |
| | IPVN | |
| Cry3Aa wild-type | ATGAATCCGAACAATCGAAGTGAACATGATACAATAAAAA | 2 |
| nucleotide | CTACTGAAAATAATGAGGTGCCAACTAACCATGTTCAATA | |
| | TCCTTTAGCGGAAACTCCAAATCCAACACTAGAAGATTTA | |
| | AATTATAAAGAGTTTTTAAGAATGACTGCAGATAATAATA | |
| | CGGAAGCACTAGATAGCTCTACAACAAAAGATGTCATTCA | |
| | AAAAGGCATTTCCGTAGTAGGTGATCTCCTAGGCGTAGTA | |
| | GGTTTCCCGTTTGGTGGAGCGCTTGTTTCGTTTTATACAA | |
| | ACTTTTTAAATACTATTTGGCCAAGTGAAGACCCGTGGAA | |
| | GGCTTTTATGGAACAAGTAGAAGCATTGATGGATCAGAAA | |
| | ATAGCTGATTATGCAAAAAATAAAGCTCTTGCAGAGTTAC | |
| | AGGGCCTTCAAAATAATGTCGAAGATTATGTGAGTGCATT | |
| | GAGTTCATGGCAAAAAAATCCTGTGAGTTCACGAAATCCA | |
| | CATAGCCAGGGGCGGATAAGAGAGCTGTTTTCTCAAGCAG | |
| | AAAGTCATTTTCGTAATTCAATGCCTTCGTTTGCAATTTC | |
| | TGGATACGAGGTTCTATTTCTAACAACATATGCACAAGCT | |
| | GCCAACACACATTTATTTTTACTAAAAGACGCTCAAATTT | |
| | ATGGAGAAGAATGGGGATACGAAAAAGAAGATATTGCTGA | |
| | ATTTTATAAAAGACAACTAAAACTTACGCAAGAATATACT | |
| | GACCATTGTGTCAAATGGTATAATGTTGGATTAGATAAAT | |
| | TAAGAGGTTCATCTTATGAATCTTGGGTAAACTTTAACCG | |
| | TTATCGCAGAGAGATGACATTAACAGTATTAGATTTAATT | |
| | GCACTATTTCCATTGTATGATGTTCGGCTATACCCAAAAG | |
| | AAGTTAAAACCGAATTAACAAGAGACGTTTTAACAGATCC | |
| | AATTGTCGGAGTCAACAACCTTAGGGGCTATGGAACAACC | |
| | TTCTCTAATATAGAAAATTATATTCGAAAACCACATCTAT | |
| | TTGACTATCTGCATAGAATTCAATTTCACACGCGGTTCCA | |
| | ACCAGGATATTATGGAAATGACTCTTTCAATTATTGGTCC | |
| | GGTAATTATGTTTCAACTAGACCAAGCATAGGATCAAATG | |
| | ATATAATCACATCTCCATTCTATGGAAATAAATCCAGTGA | |
| | ACCTGTACAAAATTTAGAATTTAATGGAGAAAAAGTCTAT | |
| | AGAGCCGTAGCAAATACAAATCTTGCGGTCTGGCCGTCCG | |
| | CTGTATATTCAGGTGTTACAAAAGTGGAATTTAGCCAATA | |
| | TAATGATCAAACAGATGAAGCAAGTACACAAACGTACGAC | |
| | TCAAAAAGAAATGTTGGCGCGGTCAGCTGGGATTCTATCG | |
| | ATCAATTGCCTCCAGAAACAACAGATGAACCTCTAGAAAA | |
| | GGGATATAGCCATCAACTCAATTATGTAATGTGCTTTTTA | |
| | ATGCAGGGTAGTAGAGGAACAATCCCAGTGTTAACTTGGA | |
| | CACATAAAAGTGTAGACTTTTTTAACATGATTGATTCGAA | |
| | AAAAATTACACAACTTCCGTTAGTAAAGGCATATAAGTTA | |
| | CAATCTGGTGCTTCCGTTGTCGCAGGTCCTAGGTTTACAG | |
| | GAGGAGATATCATTCAATGCACAGAAAATGGAAGTGCGGC | |
| | AACTATTTACGTTACACCGGATGTGTCGTACTCTCAAAAA | |
| | TATCGAGCTAGAATTCATTATGCTTCTACATCTCAGATAA | |
| | CATTTACACTCAGTTTAGACGGGGCACCATTTAATCAATA | |
| | CTATTTCGATAAAACGATAAATAAAGGAGACACATTAACG | |
| | TATAATTCATTTAATTTAGCAAGTTTCAGCACACCATTCG | |
| | AATTATCAGGGAATAACTTACAAATAGGCGTCACAGGATT | |
| | AAGTGCTGGAGATAAAGTTTATATAGACAAAATTGAATTT | |
| | ATTCCAGTGAATTAA | |
| Pos5R amino acid | MNPNNRSEHDTIKTTENNEVPTNHVQYPLAETPNPTLEDL | 3 |
| | NYKEFLRMTADNNTEALDSSTTKDVIQKGISVVGDLLGVV | |
| | GFPFGGALVSFYTNFLNTIWPSEDPWKAFMEQVEALMRQK | |
| | IADYAKNKALAELQGLQNNVEDYVSALSSWQKNPVSSRNP | |
| | HSQGRIRELFSQAESHFRNSMPSFAIRGYEVLFLTTYAQA | |
| | ANTHLFLLKDAQIYGEEWGYEKEDIAEFYKRQLKLTQEYT | |
| | DHCVKWYNVGLDKLRGRSYESWVNFNRYRREMTLTVLDLI | |
| | ALFPLYDVRLYPKEVKTELTRDVLTDPIVGVNNLRGYGTT | |
| | FSNIERYIRKPHLFDYLHRIQFHTRFQPGYYGNDSFNYWS | |
| | GNYVSTRPSIGSNDIITSPFYGNKSSEPVQNLEFNGEKVY | |
| | RAVANTNLAVWPSAVYSGVTKVQFSQYNDQTNQASTQTYD | |
| | SKRNVGAVSWDSIDQLPPETTDEPLQKGYSHQLNYVMCFL | |
| | MQGSRGTIPVLTWTHKSVDFFNMIDSKKITQLPLVKAYKL | |
| | QSGASVVAGPRFTGGDIIQCTENGSAATIYVTPDVSYSQK | |
| | YRARIHYASTSQITFTLSLDGAPFNQYYFDKTINKGDTLT | |
| | YNSFNLASFSTPFELSGNNLQIGVTGLSRGDKVYIDKIEF | |
| | IPVN | |
| Pos5R nucleotide | ATGAATCCGAACAATCGAAGTGAACATGATACAATAAAAA | 4 |
| | CTACTGAAAATAATGAGGTGCCAACTAACCATGTTCAATA | |
| | TCCTTTAGCGGAAACTCCAAATCCAACACTAGAAGATTTA | |
| | AATTATAAAGAGTTTTTAAGAATGACTGCAGATAATAATA | |
| | CGGAAGCACTAGATAGCTCTACAACAAAAGATGTCATTCA | |
| | AAAAGGCATTTCCGTAGTAGGTGATCTCCTAGGCGTAGTA | |
| | GGTTTCCCGTTTGGTGGAGCGCTTGTTTCGTTTTATACAA | |
| | ACTTTTTAAATACTATTTGGCCAAGTGAAGACCCGTGGAA | |
| | GGCTTTTATGGAACAAGTAGAAGCATTGATGCGTCAGAAA | |
| | ATAGCTGATTATGCAAAAAATAAAGCTCTTGCAGAGTTAC | |
| | AGGGCCTTCAAAATAATGTCGAAGATTATGTGAGTGCATT | |
| | GAGTTCATGGCAAAAAAATCCTGTGAGTTCACGAAATCCA | |
| | CATAGCCAGGGGCGGATAAGAGAGCTGTTTTCTCAAGCAG | |
| | AAAGTCATTTTCGTAATTCAATGCCTTCGTTTGCAATTCG | |
| | TGGATACGAGGTTCTATTTCTAACAACATATGCACAAGCT | |
| | GCCAACACACATTTATTTTTACTAAAAGACGCTCAAATTT | |
| | ATGGAGAAGAATGGGGATACGAAAAAGAAGATATTGCTGA | |
| | ATTTTATAAAAGACAACTAAAACTTACGCAAGAATATACT | |
| | GACCATTGTGTCAAATGGTATAATGTTGGATTAGATAAAT | |
| | TAAGAGGTAGATCTTATGAATCTTGGGTAAACTTTAACCG | |
| | TTATCGCAGAGAGATGACATTAACAGTATTAGATTTAATT | |
| | GCACTATTTCCATTGTATGATGTTCGGCTATATCCGAAAG | |
| | AAGTTAAAACGGAATTGACAAGAGACGTTCTTACAGATCC | |
| | AATCGTCGGTGTTAATAACCTCCGGGGTTATGGGACGACA | |
| | TTCAGCAATATCGAGAGATACATACGTAAACCTCACTTGT | |
| | TTGACTATTTACATCGCATACAGTTCCATACCCGGTTCCA | |
| | ACCTGGATACTACGGAAACGACTCTTTTAACTACTGGTCT | |
| | GGTAATTACGTCAGCACCCGGCCTAGCATAGGCTCAAACG | |
| | ATATTATCACGAGCCCTTTCTACGGAAATAAAAGCTCAGA | |
| | GCCAGTACAAAACTTAGAATTCAACGGTGAAAAGGTGTAT | |
| | AGAGCCGTTGCGAATACCAACTTAGCGGTATGGCCGAGTG | |
| | CCGTATATTCTGGTGTAACAAAGGTACAGTTTAGCCAATA | |
| | TAACGATCAGACGAACCAGGCGTCCACCCAAACATACGAC | |
| | AGCAAACGTAACGTTGGAGCAGTTTCATGGGATTCTATAG | |
| | ATCAACTTCCGCCAGAAACAACAGATGAACCACTTCAGAA | |
| | GGGTTATTCACACCAGCTCAATTACGTGATGTGTTTTCTG | |
| | ATGCAGGGATCACGCGGGACCATTCCAGTCTTGACCTGGA | |
| | CGCATAAAAGTGTAGACTTTTTCAACATGATTGATTCGAA | |
| | AAAAATTACACAACTTCCGTTAGTAAAGGCATATAAGTTA | |
| | CAATCTGGTGCTTCCGTTGTCGCAGGTCCTAGGTTTACAG | |
| | GAGGAGATATCATTCAATGCACAGAAAATGGAAGTGCGGC | |
| | AACTATTTACGTTACACCGGATGTGTCGTACTCTCAAAAA | |
| | TATCGAGCTAGAATTCATTATGCTTCTACATCTCAGATAA | |
| | CATTTACACTCAGTTTAGACGGGGCACCATTTAATCAATA | |
| | CTATTTCGATAAAACGATAAATAAAGGAGACACATTAACG | |
| | TATAATTCATTTAATTTAGCAAGTTTCAGCACACCATTCG | |
| | AATTATCAGGGAATAACTTACAAATAGGCGTCACAGGATT | |
| | AAGTCGTGGAGATAAAGTTTATATAGACAAAATTGAATTT | |
| | ATTCCAGTGAATTAA | |
| Pos8R amino acid | MNPNNRSEHDTIKTTENNEVPTNHVQYPLAETPNPTLEDL | 5 |
| | NYKEFLRMTADNNTEALDSSTTKDVIQKGISVVGDLLGVV | |
| | GFPFGGALVSFYTNFLNTIWPSEDPWKAFMEQVEALMRQK | |
| | IADYAKNKALAELQGLQNNVEDYVSALSSWQKNPVSSRNP | |
| | HSQGRIRELFSQAESHFRNSMPSFAIRGYEVLFLTTYAQA | |
| | ANTHLFLLKDAQIYGEEWGYEKEDIAEFYKRQLKLTQEYT | |
| | DHCVKWYNVGLDKLRGRSYESWVNFNRYRREMTLTVLDLI | |
| | ALFPLYDVRLYPKEVKTELTRDVLTDPIVGVNNLRGYGTT | |
| | FSNIERYIRKPHLFDYLHRIQFHTRFQPGYYGNDSFNYWS | |
| | GNYVSTRPSIGSNDIITSPFYGNKSSEPVQRLEFNGEKVY | |
| | RAVANTNLAVWPSAVYSGVTKVQFSQYNDQTNRASTQTYD | |
| | SKRNVGAVSWDSIDQLPPETTDEPLRKGYSHQLNYVMCFL | |
| | MQGSRGTIPVLTWTHKSVDFFNMIDSKKITQLPLVKAYKL | |
| | QSGASVVAGPRFTGGDIIQCTENGSAATIYVTPDVSYSQK | |
| | YRARIHYASTSQITFTLSLDGAPFNQYYFDKTINKGDTLT | |
| | YNSFNLASFSTPFELSGNNLQIGVTGLSRGDKVYIDKIEF | |
| | IPVN | |
| Pos8R nucleotide | ATGAATCCGAACAATCGAAGTGAACATGATACAATAAAAA | 6 |
| | CTACTGAAAATAATGAGGTGCCAACTAACCATGTTCAATA | |
| | TCCTTTAGCGGAAACTCCAAATCCAACACTAGAAGATTTA | |
| | AATTATAAAGAGTTTTTAAGAATGACTGCAGATAATAATA | |
| | CGGAAGCACTAGATAGCTCTACAACAAAAGATGTCATTCA | |
| | AAAAGGCATTTCCGTAGTAGGTGATCTCCTAGGCGTAGTA | |
| | GGTTTCCCGTTTGGTGGAGCGCTTGTTTCGTTTTATACAA | |
| | ACTTTTTAAATACTATTTGGCCAAGTGAAGACCCGTGGAA | |
| | GGCTTTTATGGAACAAGTAGAAGCATTGATGCGTCAGAAA | |
| | ATAGCTGATTATGCAAAAAATAAAGCTCTTGCAGAGTTAC | |
| | AGGGCCTTCAAAATAATGTCGAAGATTATGTGAGTGCATT | |
| | GAGTTCATGGCAAAAAAATCCTGTGAGTTCACGAAATCCA | |
| | CATAGCCAGGGGCGGATAAGAGAGCTGTTTTCTCAAGCAG | |
| | AAAGTCATTTTCGTAATTCAATGCCTTCGTTTGCAATTCG | |
| | TGGATACGAGGTTCTATTTCTAACAACATATGCACAAGCT | |
| | GCCAACACACATTTATTTTTACTAAAAGACGCTCAAATTT | |
| | ATGGAGAAGAATGGGGATACGAAAAAGAAGATATTGCTGA | |
| | ATTTTATAAAAGACAACTAAAACTTACGCAAGAATATACT | |
| | GACCATTGTGTCAAATGGTATAATGTTGGATTAGATAAAT | |
| | TAAGAGGTAGATCTTATGAATCTTGGGTAAACTTTAACCG | |
| | TTATCGCAGAGAGATGACATTAACAGTATTAGATTTAATT | |
| | GCACTATTTCCATTGTATGATGTTCGGCTATATCCGAAAG | |
| | AAGTTAAAACGGAATTGACAAGAGACGTTCTTACAGATCC | |
| | AATCGTCGGTGTTAATAACCTCCGGGGTTATGGGACGACA | |
| | TTCAGCAATATCGAGAGATACATACGTAAACCTCACTTGT | |
| | TTGACTATTTACATCGCATACAGTTCCATACCCGGTTCCA | |
| | ACCTGGATACTACGGAAACGACTCTTTTAACTACTGGTCT | |
| | GGTAATTACGTCAGCACCCGGCCTAGCATAGGCTCAAACG | |
| | ATATTATCACGAGCCCTTTCTACGGAAATAAAAGCTCAGA | |
| | GCCAGTACAAAGATTAGAATTCAACGGTGAAAAGGTGTAT | |
| | AGAGCCGTTGCGAATACCAACTTAGCGGTATGGCCGAGTG | |
| | CCGTATATTCTGGTGTAACAAAGGTACAGTTTAGCCAATA | |
| | TAACGATCAGACGAACAGAGCGTCCACCCAAACATACGAC | |
| | AGCAAACGTAACGTTGGAGCAGTTTCATGGGATTCTATAG | |
| | ATCAACTTCCGCCAGAAACAACAGATGAACCACTTAGAAA | |
| | GGGTTATTCACACCAGCTCAATTACGTGATGTGTTTTCTG | |
| | ATGCAGGGATCACGCGGGACCATTCCAGTCTTGACCTGGA | |
| | CGCATAAAAGTGTAGACTTTTTCAACATGATTGATTCGAA | |
| | AAAAATTACACAACTTCCGTTAGTAAAGGCATATAAGTTA | |
| | CAATCTGGTGCTTCCGTTGTCGCAGGTCCTAGGTTTACAG | |
| | GAGGAGATATCATTCAATGCACAGAAAATGGAAGTGCGGC | |
| | AACTATTTACGTTACACCGGATGTGTCGTACTCTCAAAAA | |
| | TATCGAGCTAGAATTCATTATGCTTCTACATCTCAGATAA | |
| | CATTTACACTCAGTTTAGACGGGGCACCATTTAATCAATA | |
| | CTATTTCGATAAAACGATAAATAAAGGAGACACATTAACG | |
| | TATAATTCATTTAATTTAGCAAGTTTCAGCACACCATTCG | |
| | AATTATCAGGGAATAACTTACAAATAGGCGTCACAGGATT | |
| | AAGTCGTGGAGATAAAGTTTATATAGACAAAATTGAATTT | |
| | ATTCCAGTGAATTAA | |
| Pos11R amino | MNPNNRSEHDTIKTTENNEVPTNHVQYPLAETPNPTLEDL | 7 |
| acid | NYKEFLRMTADNNTEALDSSTTKDVIQKGISVVGDLLGVV | |
| | GFPFGGALVSFYTNFLNTIWPSEDPWKAFMEQVEALMRQK | |
| | IADYAKNKALAELQGLQNNVEDYVSALSSWQKNPVSSRNP | |
| | HSQGRIRELFSQAESHFRNSMPSFAIRGYEVLFLTTYAQA | |
| | ANTHLFLLKDAQIYGEEWGYEKEDIAEFYKRQLKLTQEYT | |
| | DHCVKWYNVGLDKLRGRSYESWVNFNRYRREMTLTVLDLI | |
| | ALFPLYDVRLYPKEVKTELTRDVLTDPIVGVNNLRGYGTT | |
| | FSNIERYIRKPHLFDYLHRIQFHTRFQPGYYGNDSFNYWS | |
| | GNYVSTRPSIGSNDIITSPFYGNKSSEPVQRLEFNGEKVY | |
| | RAVANTNLAVWPSAVYSGVTKVRFSQYNDRTNRASRQTYD | |
| | SKRNVGAVSWDSIDQLPPETTDEPLRKGYSHQLNYVMCFL | |
| | MQGSRGTIPVLTWTHKSVDFFNMIDSKKITQLPLVKAYKL | |
| | QSGASVVAGPRFTGGDIIQCTENGSAATIYVTPDVSYSQK | |
| | YRARIHYASTSQITFTLSLDGAPFNQYYFDKTINKGDTLT | |
| | YNSFNLASFSTPFELSGNNLQIGVTGLSRGDKVYIDKIEF | |
| | IPVN | |
| Pos11R nucleotide | ATGAATCCGAACAATCGAAGTGAACATGATACAATAAAAA | 8 |
| | CTACTGAAAATAATGAGGTGCCAACTAACCATGTTCAATA | |
| | TCCTTTAGCGGAAACTCCAAATCCAACACTAGAAGATTTA | |
| | AATTATAAAGAGTTTTTAAGAATGACTGCAGATAATAATA | |
| | CGGAAGCACTAGATAGCTCTACAACAAAAGATGTCATTCA | |
| | AAAAGGCATTTCCGTAGTAGGTGATCTCCTAGGCGTAGTA | |
| | GGTTTCCCGTTTGGTGGAGCGCTTGTTTCGTTTTATACAA | |
| | ACTTTTTAAATACTATTTGGCCAAGTGAAGACCCGTGGAA | |
| | GGCTTTTATGGAACAAGTAGAAGCATTGATGCGTCAGAAA | |
| | ATAGCTGATTATGCAAAAAATAAAGCTCTTGCAGAGTTAC | |
| | AGGGCCTTCAAAATAATGTCGAAGATTATGTGAGTGCATT | |
| | GAGTTCATGGCAAAAAAATCCTGTGAGTTCACGAAATCCA | |
| | CATAGCCAGGGGCGGATAAGAGAGCTGTTTTCTCAAGCAG | |
| | AAAGTCATTTTCGTAATTCAATGCCTTCGTTTGCAATTCG | |
| | TGGATACGAGGTTCTATTTCTAACAACATATGCACAAGCT | |
| | GCCAACACACATTTATTTTTACTAAAAGACGCTCAAATTT | |
| | ATGGAGAAGAATGGGGATACGAAAAAGAAGATATTGCTGA | |
| | ATTTTATAAAAGACAACTAAAACTTACGCAAGAATATACT | |
| | GACCATTGTGTCAAATGGTATAATGTTGGATTAGATAAAT | |
| | TAAGAGGTAGATCTTATGAATCTTGGGTAAACTTTAACCG | |
| | TTATCGCAGAGAGATGACATTAACAGTATTAGATTTAATT | |
| | GCACTATTTCCATTGTATGATGTTCGGCTATATCCGAAAG | |
| | AAGTTAAAACGGAATTGACAAGAGACGTTCTTACAGATCC | |
| | AATCGTCGGTGTTAATAACCTCCGGGGTTATGGGACGACA | |
| | TTCAGCAATATCGAGAGATACATACGTAAACCTCACTTGT | |
| | TTGACTATTTACATCGCATACAGTTCCATACCCGGTTCCA | |
| | ACCTGGATACTACGGAAACGACTCTTTTAACTACTGGTCT | |
| | GGTAATTACGTCAGCACCCGGCCTAGCATAGGCTCAAACG | |
| | ATATTATCACGAGCCCTTTCTACGGAAATAAAAGCTCAGA | |
| | GCCAGTACAAAGATTAGAATTCAACGGTGAAAAGGTGTAT | |
| | AGAGCCGTTGCGAATACCAACTTAGCGGTATGGCCGAGTG | |
| | CCGTATATTCTGGTGTAACAAAGGTACGTTTTAGCCAATA | |
| | TAACGATCGTACGAACAGAGCGTCCCGTCAAACATACGAC | |
| | AGCAAACGTAACGTTGGAGCAGTTTCATGGGATTCTATAG | |
| | ATCAACTTCCGCCAGAAACAACAGATGAACCACTTAGAAA | |
| | GGGTTATTCACACCAGCTCAATTACGTGATGTGTTTTCTG | |
| | ATGCAGGGATCACGCGGGACCATTCCAGTCTTGACCTGGA | |
| | CGCATAAAAGTGTAGACTTTTTCAACATGATTGATTCGAA | |
| | AAAAATTACACAACTTCCGTTAGTAAAGGCATATAAGTTA | |
| | CAATCTGGTGCTTCCGTTGTCGCAGGTCCTAGGTTTACAG | |
| | GAGGAGATATCATTCAATGCACAGAAAATGGAAGTGCGGC | |
| | AACTATTTACGTTACACCGGATGTGTCGTACTCTCAAAAA | |
| | TATCGAGCTAGAATTCATTATGCTTCTACATCTCAGATAA | |
| | CATTTACACTCAGTTTAGACGGGGCACCATTTAATCAATA | |
| | CTATTTCGATAAAACGATAAATAAAGGAGACACATTAACG | |
| | TATAATTCATTTAATTTAGCAAGTTTCAGCACACCATTCG | |
| | AATTATCAGGGAATAACTTACAAATAGGCGTCACAGGATT | |
| | AAGTCGTGGAGATAAAGTTTATATAGACAAAATTGAATTT | |
| | ATTCCAGTGAATTAA | |
| SupPos3A amino | MNPNNRSEHDTIKTTENNEVPTNHVQYPLAETPNPTLEDL | 9 |
| acid | NYKEFLRMTADNNTEALDSSTTKDVIQKGISVVGDLLGVV | |
| | GFPFGGALVSFYTNFLNTIWPSEDPWKAFMEQVEALMRQK | |
| | IADYAKNKALAELQGLQNNVEDYVSALSSWQKNPVSSRNP | |
| | HSQGRIRELFSQAESHFRNSMPSFAIRGYEVLFLTTYAQA | |
| | ANTHLFLLKDAQIYGEEWGYEKEDIAEFYKRQLKLTQEYT | |
| | DHCVKWYNVGLDKLRGRSYESWVNFNRYRREMTLTVLDLI | |
| | ALFPLYDVRLYPKEVKTELTRDVLTDPIVGVNNLRGYGTT | |
| | FRNIERYIRKPHLFDYLHRIQFHTRFQPGYYGNDSFNYWS | |
| | GNYVSTRPSIGSNDIITSPFYGNKSSEPVQRLEFRGEKVY | |
| | RAVANTNLAVWPSAVYSGVTKVRFSQYNDRTRRASRQTYD | |
| | SKRNVGAVSWDSIDQLPPETTDEPLRKGYSHQLNYVMCFL | |
| | MQGSRGTIPVLTWTHKSVDFFNMIDSKKITQLPLVKAYKL | |
| | QSGASVVAGPRFTGGDIIQCTENGSAATIYVTPDVSYSQK | |
| | YRARIHYASTSQITFTLSLDGAPFNQYYFDKTINKGDTLT | |
| | YNSFNLASFSTPFELSGNNLQIGVTGLSRGDKVYIDKIEF | |
| | IPVN | |
| SupPos3A | ATGAATCCGAACAATCGAAGTGAACATGATACAATAAAAA | 10 |
| nucleotide | CTACTGAAAATAATGAGGTGCCAACTAACCATGTTCAATA | |
| | TCCTTTAGCGGAAACTCCAAATCCAACACTAGAAGATTTA | |
| | AATTATAAAGAGTTTTTAAGAATGACTGCAGATAATAATA | |
| | CGGAAGCACTAGATAGCTCTACAACAAAAGATGTCATTCA | |
| | AAAAGGCATTTCCGTAGTAGGTGATCTCCTAGGCGTAGTA | |
| | GGTTTCCCGTTTGGTGGAGCGCTTGTTTCGTTTTATACAA | |
| | ACTTTTTAAATACTATTTGGCCAAGTGAAGACCCGTGGAA | |
| | GGCTTTTATGGAACAAGTAGAAGCATTGATGCGTCAGAAA | |
| | ATAGCTGATTATGCAAAAAATAAAGCTCTTGCAGAGTTAC | |
| | AGGGCCTTCAAAATAATGTCGAAGATTATGTGAGTGCATT | |
| | GAGTTCATGGCAAAAAAATCCTGTGAGTTCACGAAATCCA | |
| | CATAGCCAGGGGCGGATAAGAGAGCTGTTTTCTCAAGCAG | |
| | AAAGTCATTTTCGTAATTCAATGCCTTCGTTTGCAATTCG | |
| | TGGATACGAGGTTCTATTTCTAACAACATATGCACAAGCT | |
| | GCCAACACACATTTATTTTTACTAAAAGACGCTCAAATTT | |
| | ATGGAGAAGAATGGGGATACGAAAAAGAAGATATTGCTGA | |
| | ATTTTATAAAAGACAACTAAAACTTACGCAAGAATATACT | |
| | GACCATTGTGTCAAATGGTATAATGTTGGATTAGATAAAT | |
| | TAAGAGGTAGATCTTATGAATCTTGGGTAAACTTTAACCG | |
| | TTATCGCAGAGAGATGACATTAACAGTATTAGATTTAATT | |
| | GCACTATTTCCATTGTATGATGTTCGGCTATATCCGAAAG | |
| | AAGTTAAAACGGAATTGACAAGAGACGTTCTTACAGATCC | |
| | AATCGTCGGTGTTAATAACCTCCGGGGTTATGGGACGACA | |
| | TTCAGAAATATCGAGAGATACATACGTAAACCTCACTTGT | |
| | TTGACTATTTACATCGCATACAGTTCCATACCCGGTTCCA | |
| | ACCTGGATACTACGGAAACGACTCTTTTAACTACTGGTCT | |
| | GGTAATTACGTCAGCACCCGGCCTAGCATAGGCTCAAACG | |
| | ATATTATCACGAGCCCTTTCTACGGAAATAAAAGCTCAGA | |
| | GCCAGTACAAAGATTAGAATTCAGAGGTGAAAAGGTGTAT | |
| | AGAGCCGTTGCGAATACCAACTTAGCGGTATGGCCGAGTG | |
| | CCGTATATTCTGGTGTAACAAAGGTAAGATTTAGCCAATA | |
| | TAACGATAGAACGAGAAGAGCGTCCAGACAAACATACGAC | |
| | AGCAAACGTAACGTTGGAGCAGTTTCATGGGATTCTATAG | |
| | ATCAACTTCCGCCAGAAACAACAGATGAACCACTTAGAAA | |
| | GGGTTATTCACACCAGCTCAATTACGTGATGTGTTTTCTG | |
| | ATGCAGGGATCACGCGGGACCATTCCAGTCTTGACCTGGA | |
| | CGCATAAAAGTGTAGACTTTTTCAACATGATTGATTCGAA | |
| | AAAAATTACACAACTTCCGTTAGTAAAGGCATATAAGTTA | |
| | CAATCTGGTGCTTCCGTTGTCGCAGGTCCTAGGTTTACAG | |
| | GAGGAGATATCATTCAATGCACAGAAAATGGAAGTGCGGC | |
| | AACTATTTACGTTACACCGGATGTGTCGTACTCTCAAAAA | |
| | TATCGAGCTAGAATTCATTATGCTTCTACATCTCAGATAA | |
| | CATTTACACTCAGTTTAGACGGGGCACCATTTAATCAATA | |
| | CTATTTCGATAAAACGATAAATAAAGGAGACACATTAACG | |
| | TATAATTCATTTAATTTAGCAAGTTTCAGCACACCATTCG | |
| | AATTATCAGGGAATAACTTACAAATAGGCGTCACAGGATT | |
| | AAGTCGTGGAGATAAAGTTTATATAGACAAAATTGAATTT | |
| | ATTCCAGTGAATTAA | |

As will be appreciated, the present disclosure provides variants of any of the polypeptides described herein, where in some instances a variant polypeptide or domain thereof comprises an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to the parental/reference sequence (e.g., the polypeptide of SEQ ID NO:1 comprising one or more amino acid substitutions of an uncharged or negatively charged amino acid for a positively charged amino acid), or a functional fragment thereof, where the variant or functional fragment retains the functionality of the parental/reference sequence, e.g., ability to form crystals and encapsulate the negatively-charged biomolecule.

In certain embodiments, Cry3Aa polypeptide variants having one or more amino acid substitutions in addition to the one or more substitutions for a positively-charged amino acid are provided. In some embodiments, the one or more amino acid substitutions comprise one or more conservative substitutions. Conservative substitutions are shown in Table 2 under the heading of “preferred substitutions.” More substantial changes are provided in Table 2 under the heading of “exemplary substitutions,” and as further described below in reference to amino acid side chain classes. The one or more additional amino acid substitutions may be introduced into a Cry3Aa polypeptide of interest and the products screened for a desired activity, e.g., retained/improved crystal formation, encapsulation, solubility, and/or the like.

TABLE 2
Amino Acid Substitutions

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gin (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

In some instances, the solubility of a Cry3Aa protein crystal of the present disclosure is 60% or greater, 65% or greater, 70% or greater, 75% or greater, 80% or greater, 85% or greater, or 90% or greater (e.g., from 60% to 95%), of the solubility of a wild-type Cry3A protein crystal under the same or similar conditions. Suitable conditions for assessing the solubility of the Cry3Aa protein crystal include any of the solubilization conditions described in the Experimental section below, e.g., a 15 mL solubilization reaction at pH 11 and a crystal concentration of 1 mg/ml.

A wide variety of negatively-charged biomolecules may be encapsulated in the Cry3Aa protein crystals of the present disclosure. In certain embodiments, the negatively-charged biomolecule is a nucleic acid. The terms “nucleic acid” and “polynucleotide” are used interchangeably herein to describe a polymer of any length, e.g., greater than about 10 bases, greater than about 20 bases, greater than about 30 bases, greater than about 40 bases, greater than about 50 bases, greater than about 75 bases, greater than about 100 bases, e.g., 200 or more bases composed of nucleotides, e.g., deoxyribonucleotides or ribonucleotides, and may be produced enzymatically or synthetically. Upon release from the Cry3Aa protein crystal, the nucleic acid can hybridize with naturally occurring nucleic acids (e.g., genomic DNA or RNA of an organism of interest, e.g., an agricultural pest) in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). An encapsulated nucleic acid may be single-stranded or double-stranded and may contain phosphodiester bonds, although any of the encapsulated nucleic acids may be nucleic acid analogs. For example, the encapsulated nucleic acid may comprise a modified internucleoside linkage, e.g., a phosphotriester linkage, a phosphorothioate (PS) linkage, a boranophosphate linkage, a phosphorodiamidate linkage, a phosphoamidate linkage, and/or a thiopho sphoramidate linkage. In some instances, the encapsulated nucleic acid comprises a modified sugar moiety, e.g., a substituted sugar moiety or a sugar surrogate. Substituted sugar moieties include furanosyls comprising substituents at the 2′-position, the 3′-position, the 5′-position and/or the 4′-position. A substituted sugar moiety may be a bicyclic sugar moiety (BNA). Sugar surrogates include morpholino, cyclohexenyl and cyclohexitol. In certain embodiments, the encapsulated nucleic acid comprises a modified sugar moiety, e.g., 2′-O-methyl, 2′-O-methoxyethyl (2′-O-MOE), 2′-O-aminopropyl, 2′-deoxy, 2′-O-propyl (2′-O-AP), 2′-O-dimethylaminoethyl (2′O-DMAOE), 2′-O-dimethylaminopropyl (2′-O-DMAP), 2′-O-dimethylaminoethyloxyethyl (2′O-DMAEOE), or 2′O—N-methylacetoamido (2′O-NMA) modification or a locked or bridged ribose conformation (e.g., LNA, cEt or ENA).

In some instances, the negatively-charged biomolecule is a nucleic acid adapted to reduce expression of a target gene by RNA interference (RNAi). The term RNA interference describes the principle of reducing the expression of a particular gene by complementary short nucleic acids, e.g., RNAs. In general, this effect can be induced after transcription by mRNA cleavage or translation repression and at the transcriptional level by transcriptional silencing. In certain embodiments, the nucleic acid adapted to reduce expression of a target gene by RNAi is a short interfering RNA (siRNA), a short hairpin RNA (shRNA), a DROSHA substrate, a Dicer substrate, a microRNA (miRNA), or a PIWI-interacting RNA (piRNA).

Accordingly, in some embodiments, the nucleic acid adapted to reduce expression of a target gene by RNAi is a short interfering RNA (siRNA). The siRNA may be an RNA:RNA hybrid, a DNA sense:RNA antisense hybrid, an RNA sense:DNA antisense hybrid, or a DNA:DNA hybrid duplexes, that can associate with a cytoplasmic multi-protein complex known as RNAi-induced silencing complex (RISC). RISC loaded with siRNA mediates the degradation of homologous mRNA transcripts. Each strand of an siRNA can be equal to or less than 35, 30, 25, 24, 23, 22, 21, or 20 nucleotides in length. In certain embodiments, the strand is at least 19 nucleotides in length. For example, each strand can be between 21 and 25 nucleotides in length. In some embodiments, the siRNA has a duplex region of 17, 18, 19, 20, 21, 22, 23, 24, or 25 nucleotide pairs, and one or more overhangs, such as one or two 3′ overhangs, of 2-3 nucleotides.

In some instances, the negatively-charged biomolecule is a single- or double-stranded RNA (e.g., configured for RNAi in an organism of interest, such as an insect), where the length of the RNA is from 50 bp to 1 kb in length, such as from 50 bp to 800 bp, 50 bp to 750 bp, 50 bp to 700 bp, 50 bp to 650 bp, 50 bp to 600 bp, 50 bp to 550 bp, or from 50 bp to 500 bp in length, e.g., from 60 bp to 500 bp in some instances.

In certain embodiments, the nucleic acid adapted to reduce expression of a target gene by RNAi is a short hairpin RNA (shRNA). shRNA is a form of hairpin RNA containing a fold-back stem-loop structure that gives rise to siRNA and is thus, likewise capable of sequence-specifically reducing expression of a target gene. Short hairpin RNAs are generally more stable and less susceptible to degradation in cellular environments than siRNAs. The stem loop structure of shRNAs can vary in stem length, typically from 19 to 29 nucleotides in length. In certain embodiments, an shRNA has a stem that is 19 to 21 or 27 to 29 nucleotides in length. In additional embodiments, the shRNA has a loop size of between 4 to 30 nucleotides in length. In some instances, the shRNA includes one or several G-U pairings in the hairpin stem to stabilize hairpins.

According to some embodiments, the nucleic acid adapted to reduce expression of a target gene by RNAi is a microRNA (miRNA). In the cytoplasm, pre-miRNAs are processed by Dicer/TRBP complex to form miRNA duplexes (18-25 nucleotides). The guide strand of the miRNA duplex is selected and loaded into the RNA-induced silencing complex (RISC) to form the miRNA-RISC complex (miRISC) while the passenger strand is degraded. Functional miRNA binds to the 3′-untranslated region (3′UTR) of targeted mRNA to perform posttranscriptional gene regulation, either to accelerate mRNA cleavage or degradation, or to repress translation.

In some instances, the negatively-charged biomolecule is an antisense oligonucleotide (ASO). ASOs are oligonucleotides typically 15-22 bases long designed in antisense orientation to the target RNA of interest. Hybridization of the ASO to the target RNA triggers RNase H cleavage of the RNA, which can prevent translation of target mRNAs.

Nucleic acids suitable for targeting an RNA of interest in an organism of interest (e.g., an agricultural pest) can routinely be designed using available target RNA sequence information (e.g., InsectBase 2.0, see Mei et al. (2021) Nucleic Acids Research 50 (D1): D1040-D1045, etc.) and guidelines, algorithms and programs known in the art (see, e.g., Aartsma-Rus et al., Mol. Ther. 17 (3): 548-553 (2009) and Reynolds et al., Nat. Biotech. 22 (3): 326-330 (2004), and Zhang et al., Nucleic Acids Res. 31 e72 (2003). Nucleic acids suitable for targeting an RNA of interest in an organism of interest (e.g., an agricultural pest) can likewise routinely be designed using available target RNA sequence information and commercially available programs (e.g., MysiRNA-Designer, AsiDesigner (Bioinformatics Research Center, KRIBB), siRNA Target Finder (Ambion), Block-iT RNAi Designer (Invitrogen), Gene specific siRNA selector (The Wistar Institute), siRNA Target Finder (GeneScript), siDESIGN Center (Dharmacon), SiRNA at Whitehead, siRNA Design (IDT), D: T7 RNAi Oligo Designer (Dudek P. and Picard D.), sfold-software, and RNAstructure 4.5); programs available over the internet such as Targetfinder (available at “bioit.org.cn/ao/targetfinder”); and commercial providers (e.g., Gene Tools, LLC).

According to some embodiments, the negatively-charged biomolecule is a nucleic acid that interferes with expression of a gene of a pest, a bacterium, a virus, a fungus, or a parasite. The term “pest” is defined herein as encompassing vectors of plant disease, human disease and/or livestock disease, unwanted species of bacteria, fungi, viruses, insects, nematodes mites, ticks or any organism causing harm during or otherwise interfering with the production, processing, storage, transport or marketing of food, agricultural commodities, wood and wood products or animal feedstuffs. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Lepidoptera and Coleoptera. Nucleic acids of the embodiments display activity against insect pests, which may include economically important agronomic, forest, greenhouse, nursery ornamentals, food and fiber, public and animal health, domestic and commercial structure, household and stored product pests. In some instances, the negatively-charged biomolecule is a nucleic acid that interferes with expression of a gene of a Hemipteran pest.

In certain embodiments, the negatively-charged biomolecule is a nucleic acid that interferes with expression of a gene of an agricultural pest. Non-limiting examples of agricultural pests include mites, aphids, whiteflies and thrips. Examples of other agricultural insect pests include diamondback moth (Plutella xylostella), cabbage armyworm (Mamestra brassicae), common cutworm (Spodoptera litura), codlingmoth (Cydia pomonella), bollworm (Heliothis zed), tobacco budworm (Heliothis virescens), gypsy moth (Lymantria dispar), rice leafroller (Cnaphalocrocis medinalis), smaller tea tortrix (Adoxophyes sp.), Colorado potato beetle (Leptinotarsa decemlineata), cucurbit leaf beetle (Aulacophora femoralis), boll weevil (Anthonomus grandis), planthoppers, leafhoppers, scales, bugs, grasshoppers, anthomyiid flies, scarabs, black cutworm (Agrotis ipsilon), cutworm (Agrotis segetum) and ants.

In addition, examples of other agricultural pests include soil pests, such as plant parasitic nematodes such as root-knot nematodes (Meloidogynidae), cyst nematodes (Heteroderidae), root-lesion nematodes (Pratylenchidae), white-tip nematode (Aphelenchoi desbesseyi), strawberry bud nematode (Nothotylenchus acris) and pine wood nematode (Bursaphelenchus xylophilus); gastropods such as slugs and snails; and isopods such as pill bugs (Armadillidium vulgare) and pill bugs (Porcellio scabe).

In some instances, the negatively-charged biomolecule is a nucleic acid that interferes with expression of a gene of an agricultural pest, where the agricultural pest is Diabrotica virgifera or Halyomorpha halys.

According to some embodiments, the negatively-charged biomolecule is a nucleic acid that interferes with expression of a gene of a pest, where the pest is Aedes aegypti. In certain embodiments, the negatively-charged biomolecule is a nucleic acid that interferes with expression of a rust gene, e.g., HXT1 or the like.

In certain embodiments, the negatively-charged biomolecule is a nucleic acid that interferes with expression of a gene of a pest, a parasite, a bacterium, a fungus, or a virus. Non-limiting examples of such genes include actin, VATPase, cytochrome P450, hemolin, hunchback, bellwether, fez2, bicoid, modsp, boule, gas8, gnbpal, gnpba3, tubulin, Sad, Ire, otk, and vitellogenin.

### Compositions and Methods of Use

Aspects of the present disclosure further include compositions. For example, provided are compositions comprising any of the Cry3Aa protein crystals of the present disclosure. In some instances, the compositions comprise the Cry3Aa protein crystals present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.), a solubilizing agent, a detergent (e.g., a non-ionic detergent such as Tween-20, etc.), a nuclease inhibitor (e.g., a ribonuclease inhibitor), a protease inhibitor, glycerol, a chelating agent, and the like may be present in such compositions.

In certain embodiments, a composition of the present disclosure is a biopesticide composition, where the Cry3Aa protein crystals (and encapsulated negatively-charge biomolecule, e.g., a nucleic that targets the gene of an agricultural pest via RNAi or the like) constitute a biopesticide. As used herein the terms “biopesticide” or “biopesticides” refer to a substance or mixture of substances intended for preventing, destroying or controlling any pest. Specifically, the term relates to substances or mixtures which are effective for treating, preventing, ameliorating, inhibiting, eliminating and/or delaying the onset of bacterial, fungal, viral, insect- or other pest-related infection or infestation, spore germination and/or hyphae growth. Also used as substances applied to crops either before or after harvest to protect the commodity from deterioration during storage and transport. As a contraction of “biological pesticides”, biopesticides include several types of pest management intervention through predatory, parasitic, or chemical relationships. In some embodiments, biopesticides refer to biologically active compounds which may be encapsulated in the Cry3Aa protein crystals of the present disclosure such as a polypeptide, a metabolite, a hormone, a pheromone, a macronutrient, a micronutrient, and a nucleic acid such as an RNA biomolecule including an antisense nucleic acid, dsRNA, shRNA, siRNA, miRNA, ribozyme, and aptamer.

The biopesticide compositions of the present disclosure may also include, in addition to the Cry3Aa protein crystals, a combination of inert components or agents, such as surfactants, adhesives, preservatives, solvents, antifoaming agents, thickeners, and the like.

A biopesticide composition may include one or more surfactants, which may be of the emulsifying or wetting type, and may be selected from anionic, nonionic, amphoteric and zwitterionic surfactants and mixtures thereof. Any surfactant known in the industry can be utilized. Examples of suitable anionic surfactants include, but are not limited to, alkyl sulfates, alkyl ether sulfates, alkaryl sulfonates, alkyl sulfosuccinates, n alkyl sarcosinates, alkyl phosphates, alkyl ether phosphates, alkyl ether carboxylates and alpha-olefin sulfonates, especially their ammonium, potassium, sodium, magnesium and mono, di and triethanolamine salts. Specific examples include dioctyl sodium sulphosuccinate (sold commercially as Aerosol® OT-B by Cytec) and ethoxylated tristyrylphenol phosphate potassium salt (sold commercially as Soprophor FLK by Rhodia).

Suitable amphoteric surfactants are those selected from the group consisting of sultaines (such as cocamidopropyl hydroxy sultaine); glycinates (such as cocoamphocarboxyglycinates); glycines (such as cocoamidopropyldimethylglycine); propionates (such as sodium lauriminodipropionate, sodium cocamphopropionate, disodium cocoamphodipropionate, and cocoamphocarboxypropionate). In addition, pseudo-amphoteric (ampholytic) surfactants such as betaines are also commonly grouped within the designation “Amphoteric” surfactants and can be used for similar purposes. Useful betaines include cocamidopropyl, coco, and oleamidopropyl.

Nonionic surfactants suitable for use in the compositions of the present disclosure may include condensation products of aliphatic (C₈-C₁₈) primary or secondary linear branched chain alcohols with alkylene oxides or phenols with alkylene oxides, usually ethylene oxide and generally having from 6 to 30 ethylene oxide groups. Suitable examples include the line of Tomadol® ethoxylates (Air Products), such as, for example, Tomadol® 23-5. Other nonionic surfactants suitable for use in the compositions of the present invention can include fatty acid alkanolamides. Representative fatty acid alkanolamides include those having CK)-C₁₈ carbons. For example, fatty acid diethanolamides such as isostearic acid diethanolamide and coconut fatty acid diethanolamide. Suitable fatty acid monoethanolamides, which may be used, include coconut fatty acid monoethanolamide and coco mono-isopropanolamide.

Semi-polar surfactants such as amine oxides are also suitable for use in the present compositions. These include N-alkyl amine oxide and N-stearyl dimethylamine oxide. A suitable N-acyl amide oxide includes N-cocamidopropyl dimethylamine oxide. The hydrophobic portion of the amine oxide surfactant may be provided by a fatty hydrocarbon chain having from about 10-21 carbon atoms.

The biopesticide compositions of the present disclosure may further comprise a thickener. A thickener may be desirable in order to keep the pesticide (e.g., Cry3As protein crystals) suspended within the composition. Examples of suitable thickeners include water-soluble polymers such as water-soluble saccharide and water-soluble synthetic polymer and inorganic powder such as silica, magnesium silicate, aluminum silicate, magnesium aluminum silicate, bentonite, smectite, hectorite and aluminum oxide. A mixture of two or more kinds of the above-mentioned thickener may be employed. Examples of water-soluble saccharides include xanthan gum, gum arabic, rhamsan gum, locust bean gum, carrageenan, welan gum, ligninsulfonic acid, starch, and carboxymethylcellulose and its salt.

The biopesticide compositions of the present disclosure may further include an anti-freezing agent, non-limiting examples of which include alcohols, diols, polyols, and combinations thereof. Suitable examples of anti-freezing agents that can be used in the present compositions include glycerol, methanol, ethylene glycol, propylene glycol, potassium acetate, calcium magnesium acetate, sorbitol, or urea.

The biopesticide compositions of the present disclosure may further include an antifoam agent (such as Antifoam SE23 from Wacker Silicones Corp. or SAG 30 from Univar Corp.).

The biopesticide compositions of the present disclosure may also include one or more preservative compounds. In some instances, the preservative compound acts to prevent corrosion of the container which holds the pesticide composition. Examples of suitable preservatives include, but are not limited to, sodium benzoate, benzoic acid, benzisothiazolinone (such as Proxel® GXL, produced by Arch Chemicals, Inc.) and potassium bicarbonate.

In some instances, the active pesticides are combined with one or more solvents (which may be, for example, water and/or an organic solvent) prior to forming a liquid biopesticide concentrate for application to a locus or area. It will be appreciated that the concentration of the Cry3Aa protein crystals contained in the biopesticide composition may need to be adjusted as necessary to account for the form in which the composition is being formulated and to ensure the biopesticide composition comprises the appropriate concentration of biopesticides as provided herein.

A biopesticide composition may be dispensed in an aqueous medium (e.g., water and/or an organic solvent) prior to its application to a locus or area where pest control is desired. All pesticide compositions that are or can be dispensed in an aqueous medium prior to application are, therefore, within the scope of the present disclosure, e.g., micro-emulsions, suspension concentrates, emulsifiable concentrates, wettable powders, water dispersible granules, capsule suspensions, emulsifiable granules, and combinations thereof.

A fogging solution may be prepared by combining the Cry3Aa protein crystals with an aqueous medium, such as water or petroleum distillates and mixing well until the composition is fully dispersed in the aqueous medium. The fogging solution is then used with fogging equipment as is known in the art, such as ultra-low volume (ULV) equipment, mechanical misting sprayers, aerosol generators, or thermal foggers.

In a further embodiment, a crop spray can be prepared by combining the Cry3Aa protein crystals with an aqueous medium, such as water or petroleum distillates and mixing well until the composition is fully dispersed in the aqueous medium. The crop spray is then used with crop spraying equipment as is known in the art, such as irrigation systems (for example, sprinkler, furrow, drip (trickle), or border irrigation systems), row crop sprayers, trailed crop sprayers, low volume mist blowers, hydraulic sprayers, compressed air sprayers, and the like.

### Methods of Use

Aspects of the present disclosure further include methods of using the Cry3Aa protein crystals and compositions of the present disclosure.

In certain embodiments, provided are methods comprising delivering Cry3Aa protein crystals of the present disclosure to a target. Targets of interest include, but are not limited to, plant cells, insect cells, worm cells, bacterial cells, fungal cells, virions, and cells of an aquatic animal. Non-limiting examples of aquatic animals include fish, shellfish, and crustaceans. In some instances, the target is any target surface to which the Cry3Aa protein crystals may be applied to a plant or a pest. For example, when the target is a plant, target surfaces include roots, bulbs, tubers, corms, leaves, flowers, seeds, stems, callus tissue, nuts, grains, fruit, cuttings, root stock, scions, harvested crops including roots, bulbs, tubers, corms, leaves, flowers, seeds, stems, callus tissue, nuts, grains, fruit, cuttings, root stock, scions, or any surface that may contact harvested crops including harvesting equipment, packaging equipment and packaging material.

According to some embodiments, provided are methods of controlling a pest, a bacterium, a virus, a fungus, or a parasite, the method comprising delivering an effective amount of a composition comprising the Cry3Aa protein crystals of the present disclosure to the pest, bacterium, virus, fungus, or parasite. According to some embodiments, the delivering comprises applying an effective amount of the composition to a crop plant. Exemplary crop plants to which the composition may be applied include vegetables such as broccoli, cauliflower, globe artichoke, peas, beans, kale, collard greens, spinach, arugula, beet greens, bok choy, chard, choi sum, turnip greens, endive, lettuce, mustard, greens, watercress, garlic chives, gai lan, leeks, Brussels sprouts, capers, kohlrabi, celery, rhubarb, cardoon, Chinese celery, lemon grass, asparagus, bamboo shoots, galangal, ginger, soybean, mung beans, urad, carrots parsnips, beets, radishes, rutabagas, turnips, burdocks, onions, shallots, leeks, garlic, green beans, lentils, and snow peas; fruits, such as tomatoes, cucumbers, squash, zucchinis, pumpkins, melons, peppers, eggplant, tomatillos, okra, breadfruit, avocado, blackcurrant, redcurrant, gooseberry, guava, lucuma, chili pepper, pomegranate, kiwifruit, grapes, cranberry, blueberry, orange, lemon, lime, grapefruit, blackberry, raspberry, boysenberry, pineapple, fig, mulberry, hedge apple, apple, rose hip, and strawberry; nuts such as almonds, pecans, walnuts, Brazil nuts, cashew nuts, horse chestnuts, macadamia nuts, Malabar chestnuts, mongongo, peanuts, pine nuts, and pistachios; tubers such as potatoes, sweet potatoes, cassava, yams, and dahlias; cereals or grains such as maize, rice, wheat, barley, sorghum, millet, oats, rye, triticale, fonio, buckwheat, and quinoa; fibers, including, for example, cotton, flax, hemp, kapok, jute, ramie, sisal, and other fibers from plants; stimulant crops, including, for example, coffee, cocoa bean, tea, mate, other plants; and pulses, including, for example, beans (including, for example, kidney, haricot, lima, butter, adzuki, mungo, golden, green gram, black gram, urd, scarlet runner, rice, moth, tepary, lablab, hyacinth, jack, winged, guar, velvet, yam, and other beans), horse-bean, broad bean, field bean, garden pea, chickpea, bengal gram, garbanzo, cowpea, black-eyed pea, pigeon pea, cajan pea, congo bean, lentil, bambara ground nut, earth pea, vetches, lupins, and other pulses. According to some embodiments, the composition is applied to a crop plant, where the crop plant is rice, maize, sugarcane, wheat, barley, soybean, potato, tomato, cucumber, lettuce, onion, cotton, rapeseed, coffee, tea, tobacco, or hops.

When the delivering comprises applying an effective amount of the composition to a crop plant, in some instances, the applying comprises spraying an effective amount of the composition on the crop plant. For example, a crop spray can be prepared by combining the Cry3As protein crystals with an aqueous medium, such as water or petroleum distillates and mixing well until the composition is fully dispersed in the aqueous medium. The crop spray may then be used with crop spraying equipment as is known in the art, such as irrigation systems (for example, sprinkler, furrow, drip (trickle), or border irrigation systems), row crop sprayers, trailed crop sprayers, low volume mist blowers, hydraulic sprayers, compressed air sprayers, and the like. In certain embodiments, a fogging solution can be prepared by combining the Cry3As protein crystals with an aqueous medium, such as water or petroleum distillates and mixing well until the composition is fully dispersed in the aqueous medium. The fogging solution may then be used with fogging equipment as is known in the art, such as ultra-low volume (ULV) equipment, mechanical misting sprayers, aerosol generators, or thermal foggers.

### Nucleic Acids and Cells

Aspects of the present disclosure further include nucleic acids encoding a Cry3Aa protein of any of the Cry3Aa protein crystals of the present disclosure. For example, according to some embodiments, a nucleic of the present disclosure encodes a Cry3Aa protein comprising one or any combination of amino acid substitutions selected from D118R, S187R, S257R, S322R, N326R, N391R, N395R, E423R, Q430R, D432R, E433R, E466R, and A629R, wherein numbering is as in SEQ ID NO: 1. The nucleic acid may encode a Cry3Aa protein comprising from 2 to 13 of such substitutions, e.g., from 5 to 11 of such substitutions. In some instances, the nucleic acid encodes a Cry3Aa protein comprising the amino acid substitutions D118R, S187R, S257R, N326R and A629R. The Cry3Aa protein may further comprise the amino acid substitutions N391R, E433R and/or E466R. The Cry3Aa protein may further comprise the amino acid substitutions E423R, Q430R and/or T436R.

Because of the knowledge of the codons corresponding to the various amino acids, availability of an amino acid sequence of a polypeptide of interest provides a description of all the polynucleotides capable of encoding the polypeptide of interest. The degeneracy of the genetic code, where the same amino acids are encoded by alternative or synonymous codons allows an extremely large number of nucleic acids to be made, all of which encode the Cry3Aa proteins disclosed herein. Thus, having identified a particular amino acid sequence, those of ordinary skill in the art could make any number of different nucleic acids by simply modifying the sequence of one or more codons in a way which does not change the amino acid sequence of the Cry3Aa protein of interest. In this regard, the present disclosure specifically contemplates each and every possible variation of polynucleotides that could be made by selecting combinations based upon the possible codon choices, and all such variations are to be considered specifically disclosed for any Cry3Aa protein disclosed herein.

The nucleotide sequences of the nucleic acids of the present disclosure may be codon-optimized. “Codon-optimized” refers to changes in the codons of the polynucleotide encoding a polypeptide to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, a nucleic acid of the present disclosure encoding a polypeptide may be codon-optimized for optimal production from the host organism selected for expression, e.g., a prokaryote such as Bacillus thuringiensis (Bt) or the like.

In some embodiments, a nucleic acid encoding a Cry3Aa protein may comprise a first domain encoding the Cry3Aa protein and a second domain encoding the negatively-charged biomolecule. For example, the second domain may encode a nucleic acid, e.g., an RNA adapted to reduce expression of a target gene by RNA interference (RNAi) (e.g., an siRNA, shRNA, miRNA, or the like). In other embodiments, the second domain encodes a negatively-charged protein.

Aspects of the present disclosure further include expression constructs comprising a nucleic acid of the present disclosure operably linked to a promoter. As used herein, an “expression construct” is a circular or linear polynucleotide (a polymer composed of naturally-occurring and/or non-naturally-occurring nucleotides) comprising a region that encodes the Cry3Aa protein operably linked to a suitable promoter, e.g., a constitutive or inducible promoter. In some embodiments, expression of the Cry3Aa protein is under the control of one or more exogenous (including heterologous) regulatory elements, e.g., promoter, enhancer, etc., present in the expression construct, and operably linked to the region encoding the Cry3Aa protein. In some embodiments, expression of the Cry3Aa protein may be controlled by one or more endogenous regulatory elements, e.g., promoter, enhancer, etc., at or near a genomic locus into which the expression construct is inserted.

When the expression construct comprises a nucleic acid comprising a first domain encoding the Cry3Aa protein and a second domain encoding a negatively-charged protein to be encapsulated in the Cry3Aa protein crystal, in some instances, the expression construct is configured to allow for polycistronic expression of Cry3Aa protein and negatively-charged protein. That is, two or more (e.g., each) of the proteins encoded by the expression construct may be expressed as separate proteins from the same promoter. In certain embodiments, the expression construct includes a ribosome skipping site to allow for polycistronic expression of two or more (e.g., each) of the protein-encoding regions. A non-limiting example of a suitable ribosome skipping site which may be incorporated into expression constructs is the P2A ribosome skipping site from porcine teschovirus.

The expression construct (e.g., vector) can be suitable for replication and integration in prokaryotes, eukaryotes, or both. The expression constructs may contain functionally appropriately oriented transcription and translation terminators, initiation sequences, and promoters useful for regulation of the expression of the nucleic acid encoding the Cry3Aa protein. The expression construct optionally contains generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression constructs which typically contain a strong promoter to direct transcription, a ribosome binding site for translational initiation, and a transcription/translation terminator, each in functional orientation to each other and to the protein-encoding sequence. The inclusion of selection markers in DNA vectors is also useful. Examples of such markers include genes specifying resistance to ampicillin, tetracycline, or chloramphenicol. Transducing cells with nucleic acids can involve, for example, incubating lipidic microparticles containing nucleic acids with cells or incubating viral vectors containing nucleic acids with cells within the host range of the vector.

In certain embodiments, upon delivery of the expression construct into a population of cells of interest, the expression construct is episomal (e.g., extra-chromosomal), where by “episome” or “episomal” is meant a polynucleotide that replicates independently of the cell's chromosomal DNA. A non-limiting example of an episome that may be employed is a plasmid.

According to some embodiments, upon delivery of the expression construct into a population of cells of interest, the expression construct integrates into the genome of the cell. In certain embodiments, the expression construct is adapted for site-specific integration into the genome. For example, an expression construct may be adapted for site-specific integration into the genome, where the site-specific integration inactivates a target gene within the genome of the cell. By way of example, the site-specific integration may knock-out the target gene by knock-in of the expression construct. Any suitable approach for site-specific gene editing and functional integration may be employed. Functional integration of an expression construct may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the cells in a suitable transduction medium, at a suitable concentration (or multiplicity of infection), and for a suitable time for the vector to infect the target cells, facilitating functional integration of the expression construct. Non-limiting examples of useful viral vectors include retroviral vectors, lentiviral vectors, adenoviral (Ad) vectors, adeno-associated virus (AAV) vectors, hybrid Ad-AAV vector systems, and the like.

Strategies for site-specific integration that find use include those that employ homologous recombination, nonhomologous end-joining (NHEJ), and/or the like. Such strategies may employ a non-naturally occurring or engineered nuclease, including, but not limited to, zinc-ringer nucleases (ZNFs), meganucleases, transcription activator-like effector nucleases (TALENs)), or a CRISPR-Cas system. Eukaryotic cells utilize two distinct DNA repair mechanisms in response to DNA double strand breaks (DSBs): Homologous recombination (HR) and nonhomologous end-joining (NHEJ). Mechanistically, HR is an error-free DNA repair mechanism because it requires a homologous template to repair the damaged DNA strand. Because of its homology-based mechanism, HR has been used as a tool to site-specifically engineer the genome. Gene targeting by HR requires the use of two homology arms that flank the transgene/target site of interest. HR efficiency can be increased by the introduction of DSBs at the target site using specific rare-cutting endonucleases. The discovery of this phenomenon prompted the development of methods to create site-specific DSBs in the genome of different species. Various chimeric enzymes have been designed for this purpose over the last decade, namely ZFNs, meganucleases, and TALENs. ZFNs are modular chimeric proteins that contain a ZF-based DNA binding domain (DBD) and a FokI nuclease domain. DBD is usually composed of three ZF domains, each with 3-base pair specificity; the FokI nuclease domain provides a DNA nicking activity, which is targeted by two flanking ZFNs. Owing to the modular nature of the DBD, any site in a genome could be targeted. TALENs are similar to ZFNs except that the DBD is derived from transcription activator-like effectors (TALEs). The TALE DBD is modular, and it is composed of 34-residue repeats, and its DNA specificity is determined by the number and order of repeats. Each repeat binds a single nucleotide in the target sequence through only two residues.

The expression construct may be delivered to any cell population of interest. In certain embodiments, the population of cells is a population of prokaryotic cells. In some instances, the population of cells is a population of Bacillus thuringiensis (Bt) cells. According to some embodiments, expression of the Cry3Aa protein (and optionally, the negatively-charged biomolecule) is under the control of a Cry3Aa promoter. The Cry3Aa promoter may be an exogenous Cry3Aa promoter (that is, part of the expression construct introduced into the cell) or an endogenous Cry3Aa promoter.

Accordingly, aspects of the present disclosure further include a cell or population of cells comprising any of the nucleic acids or expression constructs of the present disclosure. In certain embodiments, the population of cells is a population of prokaryotic cells, non-limiting examples of which include Bacillus thuringiensis (Bt) cells.

A variety of suitable approaches and conditions for the delivery of the expression construct to cells are known. According to some embodiments, the expression construct is delivered to the population of cells by microinjection, transfection, lipofection, heat-shock, electroporation, transduction, gene gun, DEAE-dextran-mediated transfer, and/or the like.

### Methods of Producing Cry3Aa Protein Crystals

Aspects of the present disclosure further include methods of producing Cry3Aa protein crystals. According to some embodiments, the methods comprise producing the Cry3Aa protein crystals of the present disclosure.

In certain embodiments, provided are methods of producing Cry3Aa protein crystals comprising a negatively-charged biomolecule encapsulated therein. Such methods comprise incubating a cell comprising an expression construct of the present disclosure under conditions in which Cry3Aa protein crystals are produced, and encapsulating a negatively-charged biomolecule in the Cry3Aa protein crystals.

Encapsulation of the negatively-charged biomolecule may be “direct” meaning that the encapsulation comprises combining isolated Cry3Aa protein crystals produced during the incubating step with the negatively-charged biomolecule under conditions in which the negatively-charged biomolecule is encapsulated in the Cry3Aa protein crystals. An exemplary approach for direct encapsulation of the negatively-charged biomolecule is described in detail in the Experimental section below.

In other embodiments, an “encapsulation by recrystallization” approach is employed to encapsulate the negatively-charged biomolecule. Such an approach is described for the first time herein and was determined by the inventors to dramatically increase loading of the negatively-charged biomolecule per crystal with higher negatively-charged biomolecule payloads. In the proof-of-concept example of siRNA, the encapsulation by recrystallization approach resulted in an 11-fold improvement in siRNA loading compared to the direct encapsulation approach, while still retaining high encapsulation efficiency.

In some instances, encapsulation by recrystallization comprises isolating the Cry3Aa protein crystals produced during the incubating step, solubilizing the isolated Cry3Aa protein crystals to produce solubilized Cry3Aa protein, and recrystallizing the solubilized Cry3Aa protein in the presence of the negatively-charged biomolecule. According to some embodiments, solubilizing the isolated Cry3Aa protein crystals comprises incubating the isolated Cry3Aa protein crystals in an alkaline buffer (e.g., NaCO₃ or other suitable alkaline buffer). In certain embodiments, the pH of the alkaline buffer is from 10 to 12 or from 10.5 to 11.5, optionally about pH 11. In some instances, recrystallizing the solubilized Cry3Aa protein in the presence of the negatively-charged biomolecule comprises adding an acidic buffer comprising the negatively charged biomolecule to the alkaline buffer comprising the solubilized Cry3Aa protein. According to some embodiments, the concentration of the solubilized Cry3Aa protein in the alkaline buffer is from 0.5 to 1.5 mg/ml or from 0.75 to 1.25 mg/mL, optionally about 1.0 mg/mL. In certain embodiments, the concentration of the negatively charged biomolecule in the acidic buffer is from 0.5 to 3 M. In some instances, the pH of the acidic buffer is from 3 to 5 or from 3.5 to 4.5, optionally about pH 4. In some instances, the acidic buffer is a citrate buffer. According to some embodiments, the volume of added acidic buffer provides a ratio of acidic buffer to alkaline buffer of from 3:1 to 5:1 or from 3.5:1 to 4.5:1, optionally about 4:1.

An exemplary protocol for encapsulation by recrystallization is described in detail in the Experimental section below. According to one particular non-limiting approach, for solubilization, the protein crystal may be spun down in a centrifuge at 8,000 RPM for 5 minutes. The supernatant may be removed followed by addition of pH 11, 0.1 M NaCO₃ so the protein crystal concentration is about 1 mg/mL. The protein crystal pellet may then be resuspended and incubated overnight at room temperature at 750 RPM. The next day, the solubilized crystal may be spun down at 8,500 RPM for 15 minutes. The supernatant may be filtered using a 0.45 μm syringe filter. The concentration may then be measured via the Bradford assay with the spectrophotometer set to a wavelength of 595 nm. According to one particular non-limiting approach, for recrystallization, solubilized Cry3Aa protein (e.g., solubilized as described above) is brought to a concentration of about 1 mg/mL in alkaline buffer, and a stock of negatively-charged biomolecule in acidic buffer (e.g., pH of about 4) at a concentration of from 0.5 to 3 μM is added to the solubilized Cry3Aa protein at a ratio of about 4:1, thereby reducing the pH of the Cry3Aa protein environment and recrystallizing the Cry3Aa proteins in the presence of the negatively-charged biomolecule, thereby encapsulating the negatively-charged biomolecule in Cry3Aa protein crystals. With the benefit of the present disclosure, one of ordinary skill in the art can readily optimize the recrystallization conditions for the particular negatively-charged biomolecule of interest.

As will be appreciated with the benefit of the present disclosure, the encapsulation by recrystallization approach is not limited to the engineered Cry3Aa protein crystals of the present disclosure, but rather may be employed to encapsulate any molecule of interest in any Cry3Aa protein crystal of interest, including but not limited to wild-type Cry3Aa protein crystals. For example, provided are methods of encapsulating a biomolecule in a Cry3Aa protein crystal, the methods comprising solubilizing Cry3Aa protein crystals to produce solubilized Cry3Aa protein, and recrystallizing the solubilized Cry3Aa protein in the presence of the biomolecule to be encapsulated. Exemplary solubilization and recrystallization conditions include those described above in the context of the engineered Cry3Aa protein crystals of the present disclosure and negatively-charged biomolecules.

The following examples are offered by way of illustration and not by way of limitation.

### EXPERIMENTAL

### Example 1—Modification of the Cry3Aa Crystal Solvent Channel for Encapsulation of Negatively-Charged Biomolecules

Although the solvent channels within the Cry3Aa crystal are large enough to accommodate negatively charged biomolecules (e.g., nucleic acids such as DNA or RNA (e.g., siRNAs, miRNAs, shRNAs, piRNAs, or the like)), the charge of this channel is slightly negative, which would repel the negatively-charged biomolecules. Described in this example is the development of mutant Cry3Aa proteins which form crystals having solvent channels capable of encapsulating negatively-charged biomolecules. As proof of concept, siRNAs were employed in the examples herein as the negatively-charged biomolecule.

A plasmid was first modified to recombinantly produce an engineered variant of Cry3Aa containing 14 positively charged arginines in the solvent channel. This new protein variant was designated super positive Cry3Aa (SupPos3A) and included the following amino acid substitutions: D118R, S187R, S257R, S322R, N326R, N391R, N395R, E423R, Q430R, D432R, E433R, T436R, E466R, A629R.

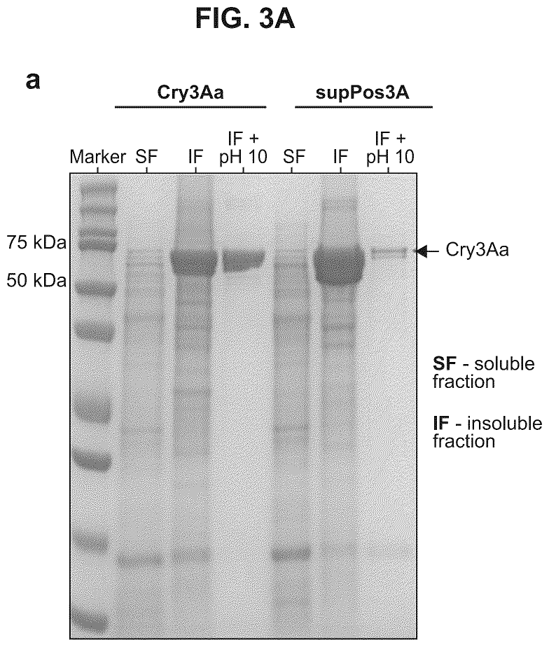

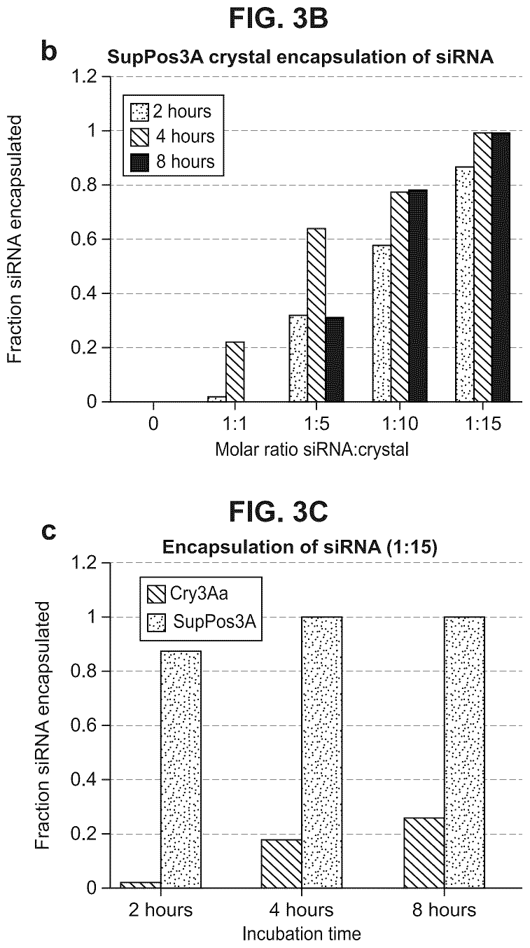

With the expression plasmid in hand, the production strain was generated and the SupPos3A crystals were produced and purified as demonstrated by SDS-PAGE (FIG. 3a). Encapsulation of the siRNA was performed by directly mixing the SupPos3A crystals with the siRNA molecule at different molar ratios. The data highlights the ability of the SupPos3A crystals to encapsulate the siRNA, where nearly 100% of the siRNA was encapsulated after 4 hours (FIG. 3b), and fine tuning of the composition of the solvent channel was crucial to direct the biomolecule of interest inside the cavity. This is demonstrated in FIG. 3c where only the modified Cry crystal encapsulated the siRNA with reasonable efficiency.

### Example 2—Encapsulation of Negatively-Charged Biomolecules by Recrystallization

Described in this example is the development of a new strategy for negatively-charged biomolecule encapsulation with higher loading of the negatively-charged biomolecule per crystal. With the direct encapsulation method up to 100% encapsulation was observed, but this was at a 1:15 siRNA: SupPos3A molar ratio, and increased encapsulation using less crystal could not be obtained albeit attempting longer incubation times. However, calculations suggested that based on the volume of siRNA and volume of the solvent channels, much more siRNA could theoretically fit inside the channel. This suggested that the crystal was reaching saturation and the siRNA was most likely surface bound, or at least not able to reach all of the solvent channel sites with the crystal.

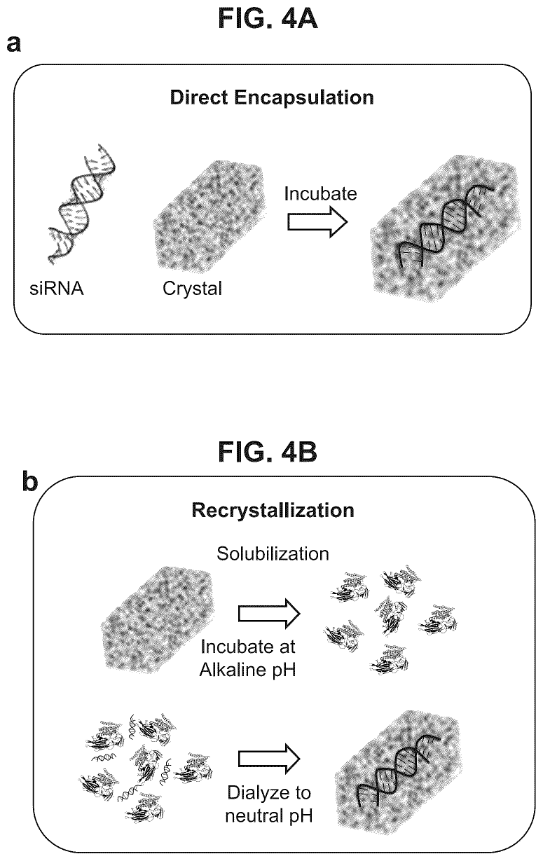

In light of these observations, other strategies for siRNA encapsulation within the Cry crystal were considered. In particular, it was surmised that if the crystals produced from bacteria could be solubilized, and then recrystallized in the presence of siRNA, more of the solvent channels within the crystals could be filled. This new approach was termed encapsulation by recrystallization, and an illustration of this approach as well as the direct encapsulation approach is illustrated in FIG. 4.

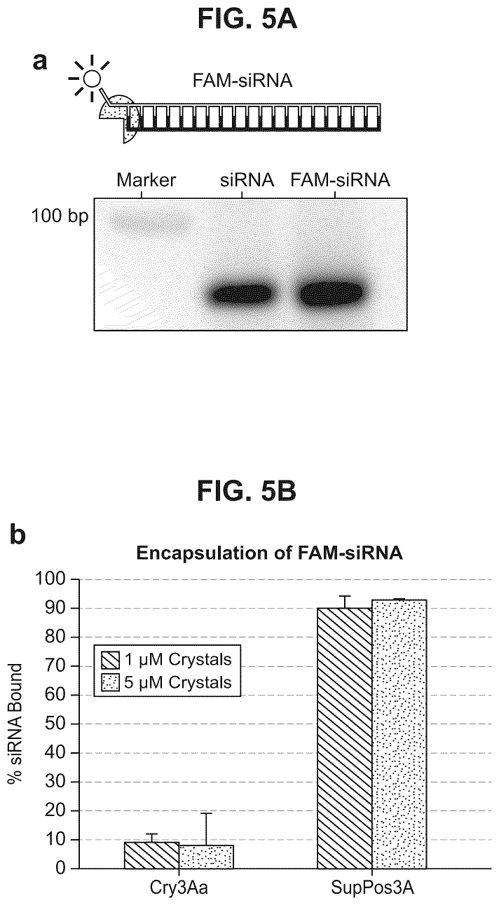

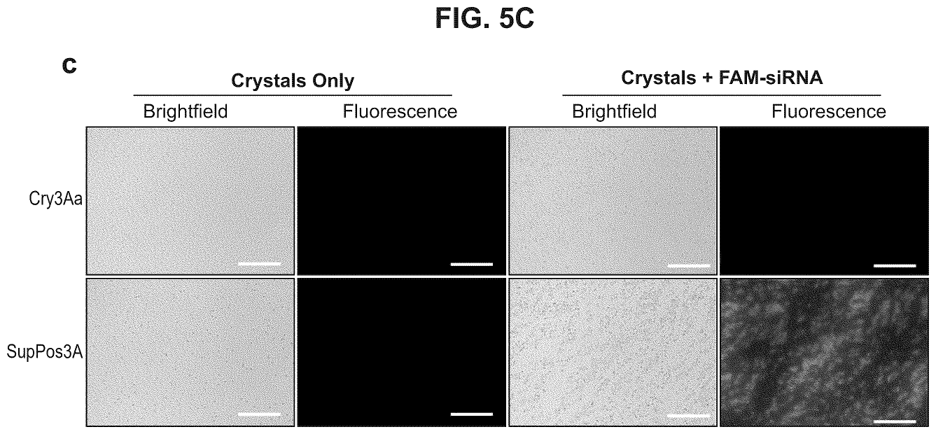

It was anticipated that many different conditions might need to be tested to optimize the recrystallization approach, and the agarose gel assay used to measure encapsulation efficiency is low throughput. Therefore, a fluorescent-based assay was developed to assess encapsulation efficiency in a higher-throughput manner. This was accomplished by labeling one of the two RNA strands within the siRNA molecule with a fluorescent dye, so that the siRNA could be tracked by monitoring fluorescence. Synthesis of the fluorescently labeled siRNA FAM-siRNA was performed and both the labeled and unlabeled siRNA molecules were compared on the agarose gel (FIG. 5a). Both ran at the same size, indicating the FAM group did not disrupt siRNA formation. Additionally, encapsulation was performed with FAM-siRNA under optimal conditions using the direct binding approach, which resulted in ˜90% encapsulation efficiency and agreed well with the agarose gel data (FIG. 5b). This approach indirectly measures the encapsulation efficiency since the fluorescence in the supernatant was measure before and after binding. As a more direct approach, fluorescence microscopy was used to examine whether the crystals were loaded with siRNA. Indeed, only SupPos3A crystals loaded with FAM-siRNA were fluorescent, indicating the siRNA is loaded on the crystal (FIG. 5c).

With a fluorescence assay developed, the recrystallization approach was evaluated. The first step to this approach is to solubilize the crystals extracted from Bt in an alkaline buffer. In initial attempts to solubilize SupPos3A, it was determined to not solubilize as well as Cry3Aa, and the results were inconsistent (some batches of crystals would solubilize better than other batches). It was postulated that the multiple arginine mutations present within the solvent channel of SupPos3A could be affecting the natural solubility of the Cry3Aa protein. Therefore, three new Cry3Aa protein variants were generated with increasing numbers of arginine residues within the solvent channel, including a total of 5 arginines (Pos5R: D118R, S187R, S257R, N326R, E423Q, D432N, E433Q, E466Q, A629R), 8 arginines (Pos8R: D118R, S187R, S257R, N326R, N391R, E423Q, D432N, E433R, E466R, A629R) and 11 arginines (Pos11R: D118R, S187R, S257R, N326R, N391R, E423R, Q430R, D432N, E433R, T436R, E466R, A629R), and SupPos3A has 14 total arginines. Plasmids containing these new constructs were transformed into Bt and crystals were expressed and purified as before.

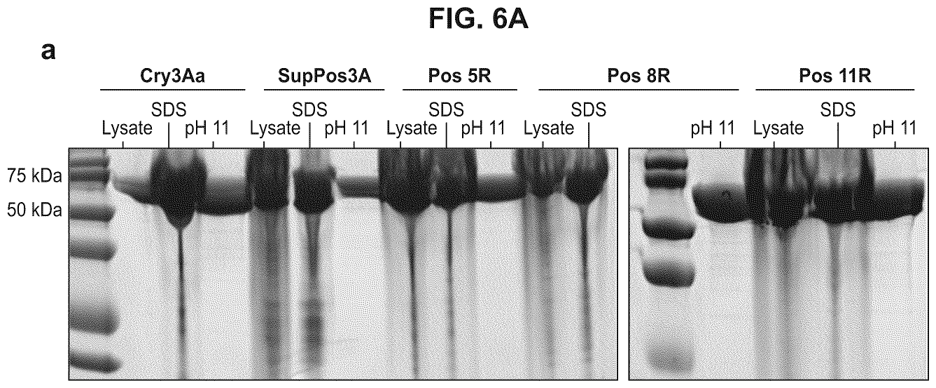

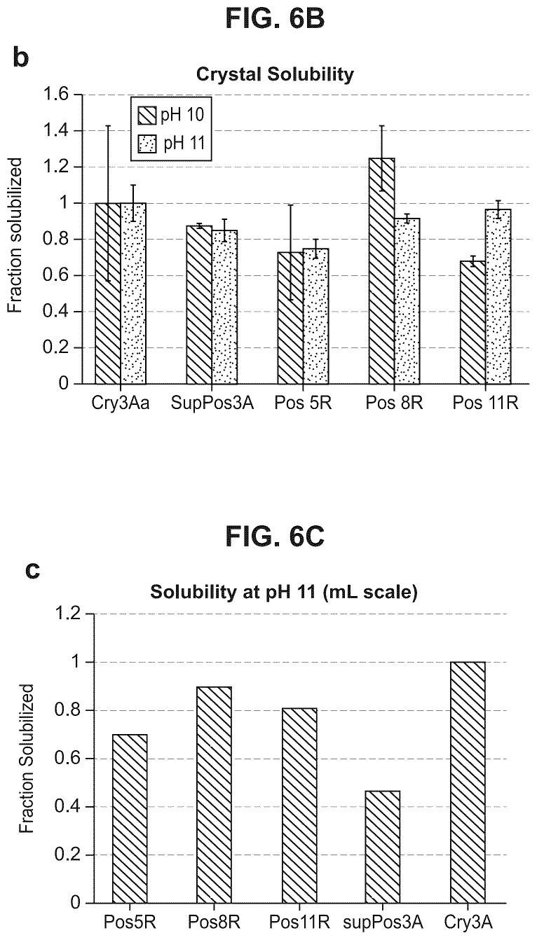

After purification, all crystals were solubilized and assessed by SDS-PAGE, which confirmed production of each crystal variant (FIG. 6a). Surprisingly, yields of SupPos3A were about 50% of the other mutants (data not shown), suggesting that too many mutations could negatively affect the yield of the crystal protein. The solubility at small scale (100 μl) was subsequently evaluated, and it was found that all constructs demonstrated similar solubility behavior (FIG. 6b). However, when the solubilization reaction was scaled up to 15 mL, a significant difference between the various constructs was observed (FIG. 6c). SupPos3A was the worst of them all, and less than 50% of the crystal solubilized at pH 11. None of the other arginine variants were as soluble as wild-type Cry3Aa, but they ranged from 70%-90% solubilization.

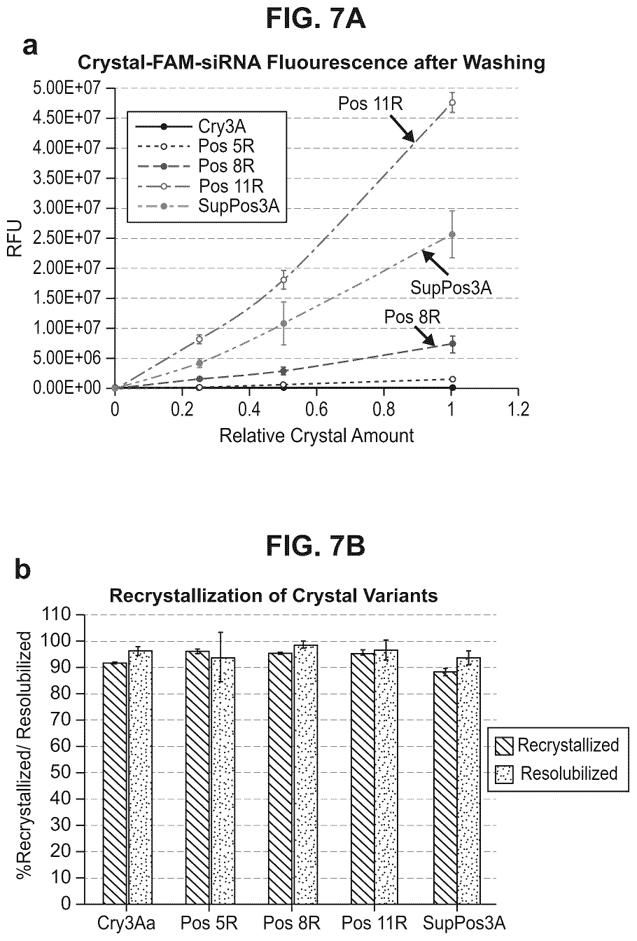

After testing solubilization of the different crystal variants, encapsulation of each variant was tested using the direct encapsulation approach. In this assay, the different crystals were incubated with FAM-siRNA, then washed several times, and crystal fluorescence was directly ascertained in a plate reader. The data from this experiment is shown in FIG. 7a. The results suggest that increasing the number of positively charged residues within the solvent channel increases the amount of siRNA bound to the crystal, with Pos5R being more fluorescent than Cry3Aa, Pos8R being more fluorescent than Pos5R, and Pos11R being more fluorescent than Pos8R. However, this trend stops at Pos11R, since SupPos3A (14R) crystals are actually less fluorescent than Pos11R. This result was unexpected and may be due to SupPos3A forming a slightly different crystal structure that reduces the number of potential siRNA binding sites compared to Pos11R.

Nevertheless, all constructs were solubilized in pH 11 buffer and a recrystallization assay was performed without siRNA, to ensure that the general approach was working. Instead of dialyzing the soluble protein variants to a neutral buffer, it was thought to be more practical to mix with an acidic buffer, in this case sodium citrate, to bring the pH to near neutral, which would cause the proteins to spontaneously recrystallize quickly. Indeed, all proteins recrystallized after addition of citrate buffer within 5 minutes. The recrystallization efficiencies for all proteins except SupPos3A was >90%, meaning >90% of the protein solubilized was recovered in crystal form (FIG. 7b). SupPos3A recrystallized at an efficiency only slightly less than 90%. To ensure the recrystallized crystals were still soluble at alkaline pH (which is critical for siRNA release within the insect midgut), the crystals were resolubilized and the protein content was determined. The data in FIG. 7b demonstrates that all recrystallized crystals retained their ability to solubilize at alkaline pH, and all solubilization efficiencies were >90%.

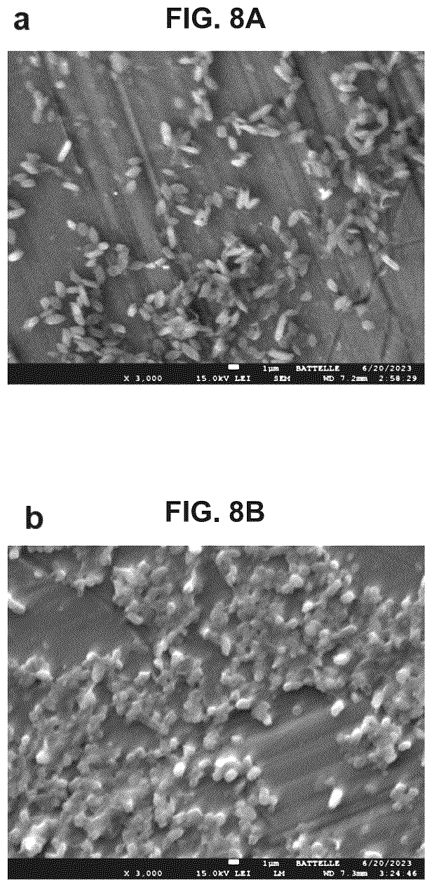

Based on the data from the second generation of positively charged mutants, Pos11R was selected as the optimal candidate for screening siRNA encapsulation by recrystallization. Pos11R crystals were produced in high yield, were highly soluble, could bind the most siRNA the during the direct encapsulation assays, and could be recrystallized and resolubilized at efficiencies of 96% and 97%, respectively. The Pos11R crystals produced in Bt. were further characterized by scanning electron microscopy (SEM) analysis, revealing that wild-type Cry3Aa crystals have a rhombus shape with sharp edges and were about 1-2 μm in length (FIG. 8a). Pos11R crystals were also 1-2 μm in length but had a more spherical shape (FIG. 8b).

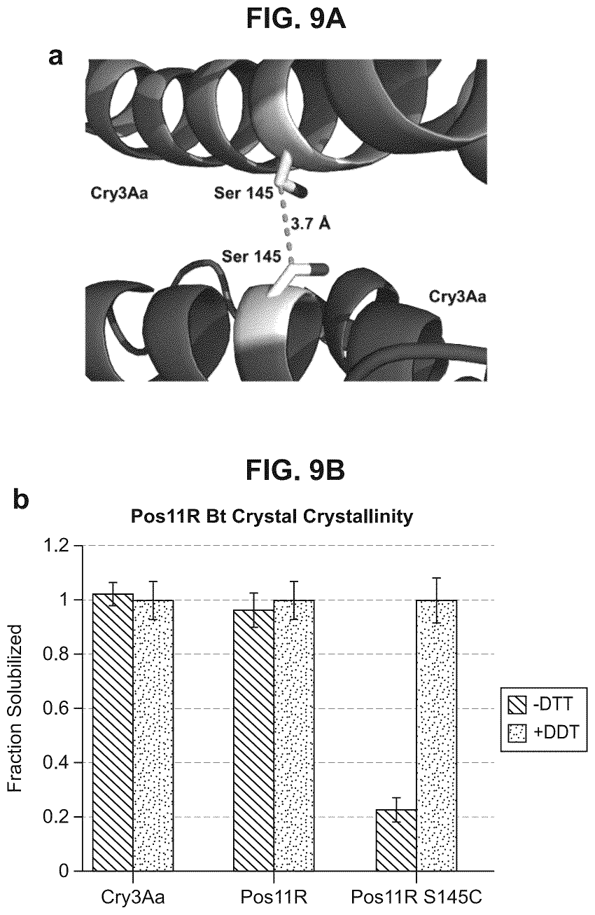

It was unclear whether the Pos11R crystals still form the same crystal lattice structure as wild-type Cry3Aa, since this would indicate that the large solvent channels for siRNA encapsulation were retained. It is tedious to generate protein crystals suitable for X-ray diffraction and subsequent structure determination, so a molecular approach was employed to determine whether the same crystal lattice was retained. When two Cry3Aa protein molecules come together in the protein crystal, serine 145 is 3.7 Å away from another serine 145 on another Cry3Aa monomer at the crystal interface, suggesting they are at an optimal distance to form a disulfide bond (FIG. 9a). This serine was mutated to a cysteine in Pos11R making Pos11R S145C, and these crystals were produced in Bt.

It was hypothesized that if the Pos11R proteins formed the same crystal lattice as wild-type Cry3Aa, the S145C mutant would form a disulfide bond, which is covalent and would render the crystals insoluble at alkaline pH. Intriguingly, the Pos11R S145C crystals were much more resistant to solubilization than Cry3Aa and Pos11R at pH 11, and when the reducing agent dithiothreitol (DTT) was added, the Pos11R S145C crystals became completely soluble (FIG. 9b). DTT had no effect on the solubilization behavior of Cry3Aa or Pos11R, which strongly suggests that the disulfide bond formed in the Pos11R S145C crystals. This disulfide bond could only occur if Pos11R formed the same crystal lattice structure as wild-type Cry3Aa, indicating that the large solvent channels are present within the Pos11R mutant crystals.

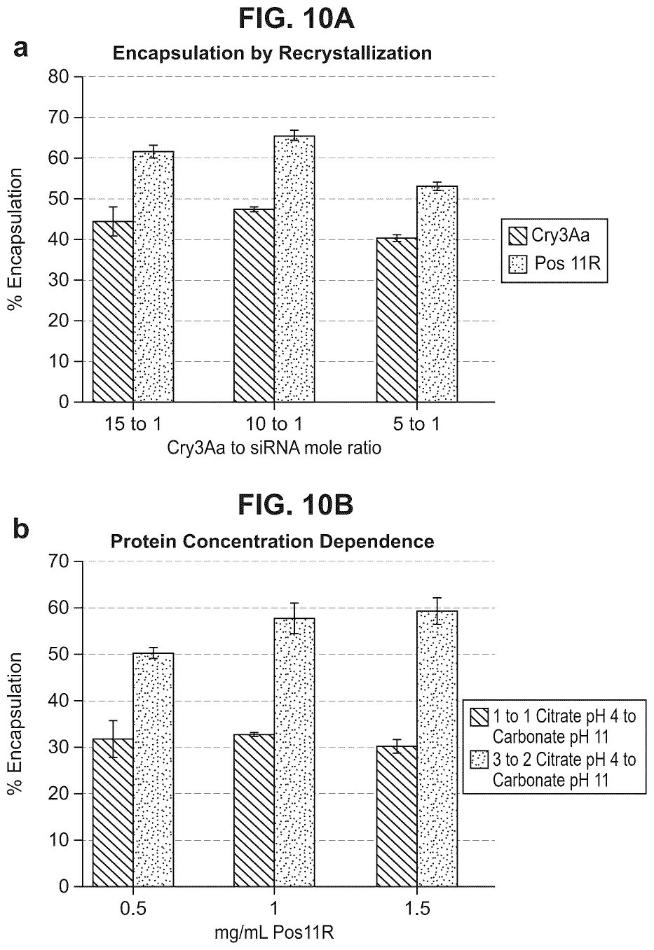

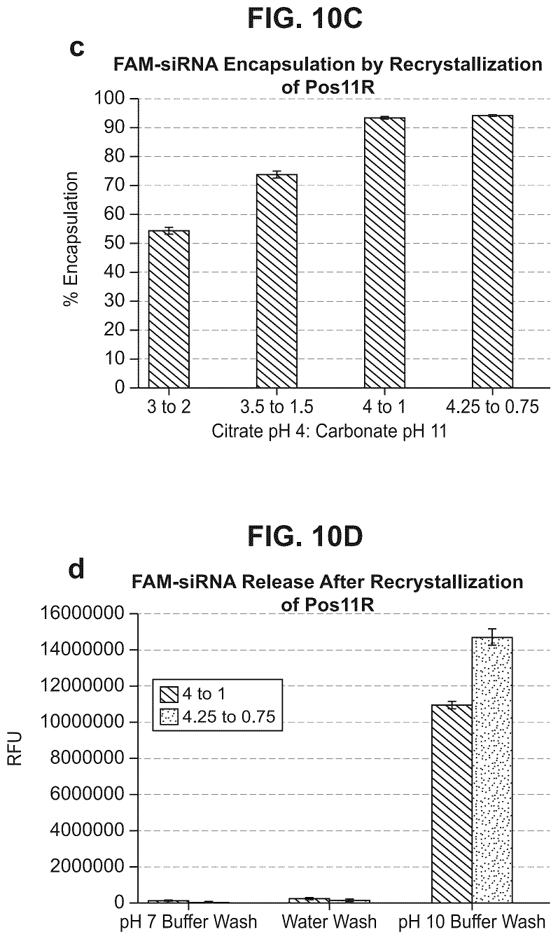

Given the aforementioned data indicating that Pos11R still forms the solvent channels capable of accommodating siRNA, and with a set of conditions for recrystallization in hand, encapsulation of siRNA was tested via the recrystallization method. This was initially performed by mixing soluble Pos11R protein in pH 11.0 buffer with FAM labeled siRNA in pH 4.0 buffer, where mixing at a 3:2 pH 4 buffer:pH 11 buffer ratio would initiate recrystallization and presumably encapsulation of the siRNA. Initial attempts yielded poor encapsulation efficiencies at all Pos11R: siRNA molar ratios examined (FIG. 10a), however, a dramatic decrease in encapsulation efficiency (% siRNA removed from supernatant) was not observed as the amount of siRNA was increased in the reaction, suggesting that the loading of siRNA per crystal was in fact increasing with higher siRNA payloads. This observation suggested that volume within the crystal was not limiting the efficiencies, and that tuning the reaction conditions could potentially improve the encapsulation efficiency at higher siRNA payloads.

Next, two more test criteria were examined: Pos11R protein concentration and buffer ratio. Here, 0.5, 1.0 and 1.5 mg/mL Pos11R was used, and a 3:2 pH 4 buffer:pH 11 ratio employed previously, and a 1:1 pH 4 buffer:pH 11 ratio were evaluated (FIG. 10b). This experiment was performed at a constant 10:1 Pos11R:siRNA molar ratio. Protein concentration had a modest effect on encapsulation efficiency, but this plateaued at 1 mg/ml Pos11R protein. However, a significant change in encapsulation efficiency was observed from a 1:1 buffer ratio to a 3:2 buffer ratio, which nearly doubled the encapsulation efficiency. This suggested that the buffer ratio has a large impact on encapsulation efficiency, so further tuning of this parameter could improve loading of the siRNA via the recrystallization method.

Increasing the amount of pH 4 buffer drastically improved the encapsulation efficiency, reaching >90% encapsulation at a 4:1 and 4.25:0.75 pH 4:pH 11 buffer ratio (FIG. 10c). Crystals with the highest siRNA loadings (4:1 and 4.25:0.75 pH 4:pH 11) were examined for stability under different conditions. These siRNA loaded crystals were washed in pH 7 buffer and water, and minimal fluorescence was detected in the supernatant (FIG. 10d). However, when these crystals were incubated at pH 10, a condition that mimics the midgut environment of insects, a significant amount of fluorescence was detected in the supernatant (FIG. 10d), indicating FAM-siRNA was released from the crystals. This data highlights the utility of this platform for siRNA delivery, since the crystals would remain stable during digestion, but a pH shift would release the siRNA cargo at the site of action.

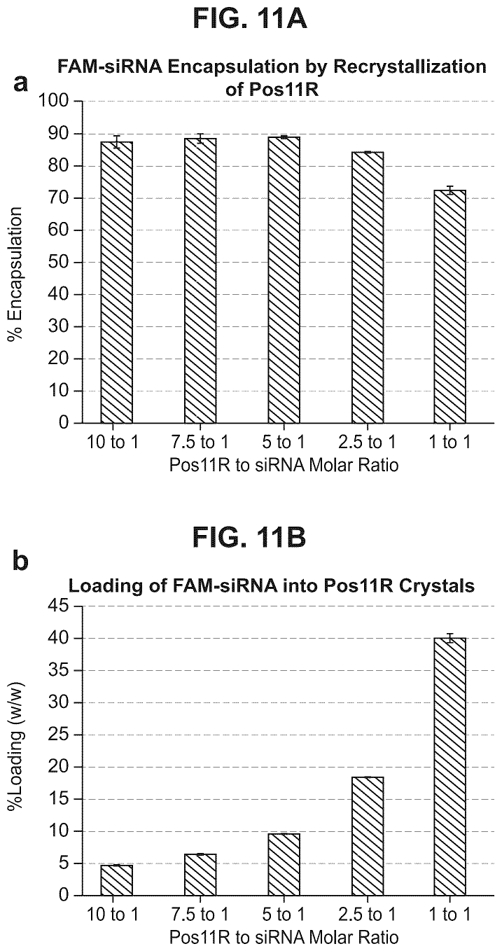

Given the promising results using a 4:1 pH 4:pH 11 buffer ratio, attempted next was to increase the loading of siRNA within the Pos11R crystal by adjusting the Pos11R:siRNA molar ratios. In the previous experiments, a 10:1 Pos11R:siRNA ratio was used, but in this experiment, a 7.5:1, 5:1, 2.5:1 and 1:1 Pos11R:siRNA ratio was evaluated using the optimal protein concentration and buffer ratio conditions identified from the previous set of experiments (1 mg/mL Pos11R and 4:1 pH 4:pH 11 buffer ratio). Under optimal conditions, observed was a ˜90% encapsulation efficiency up to a 5:1 Pos11R:siRNA ratio, a slight decrease at a 2.5:1 ratio, and still >70% at the highest siRNA loading at a 1:1 Pos11R:siRNA ratio (FIG. 11a). Converting this data into a % loading of siRNA into the crystal (i.e., g siRNA/g Pos11R), up to a 40% w/w loading was achieved (FIG. 11b), which is significantly higher than many polymer-based encapsulation formulations. Overall, an 18-fold improvement in siRNA loading was seen compared to the initial recrystallization test, and an 11-fold improvement compared to the direct encapsulation approach.

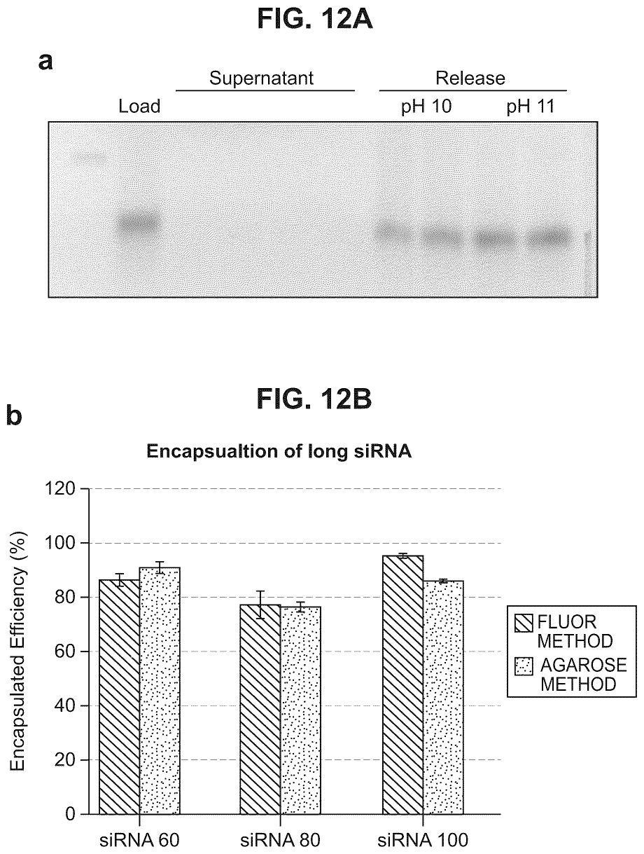

It was important to ensure that the FAM label on the siRNA was not driving the high encapsulation efficiencies and % loadings of siRNA into the Pos11R crystals following recrystallization, since the FAM group would likely not be in the final siRNA product when employing in an application of interest, e.g., in the field as a biopesticide. As such, an agarose gel-based method was used, where the unlabeled siRNA is electrophoresed on an agarose gel and stained with ethidium bromide pre- and post-recrystallization. This reaction was performed at a 2.5:1 Pos11R:siRNA ratio under optimal conditions for recrystallization. As demonstrated in FIG. 12a, barely any siRNA is visible in the supernatant following recrystallization, but after incubation in alkaline buffer pH 10 or pH 11, the unlabeled siRNA could be released just as before. Using this method, the encapsulation efficiency was calculated using densitometry analysis of the bands, which resulted in a 95.9±1.5% encapsulation efficiency.

The siRNA size constraints for encapsulation were assessed next, since longer siRNAs (100 base pairs (bp)) were shown to be more efficacious than shorter ones. All previous experiments were performed with 60 bp siRNA molecules, so tested here was encapsulation of 80 bp and 100 bp FAM-siRNA, and both the fluorescence-based method and agarose gel method were used to determine encapsulation efficiencies. Employed were the same conditions as before at a 2.5:1 Pos11R:siRNA ratio. Surprisingly, the data demonstrates that siRNA size had minimal effect on encapsulation efficiency, and that both methods to determine encapsulation efficiency gave similar results, ensuring confidence in the data (FIG. 12b).

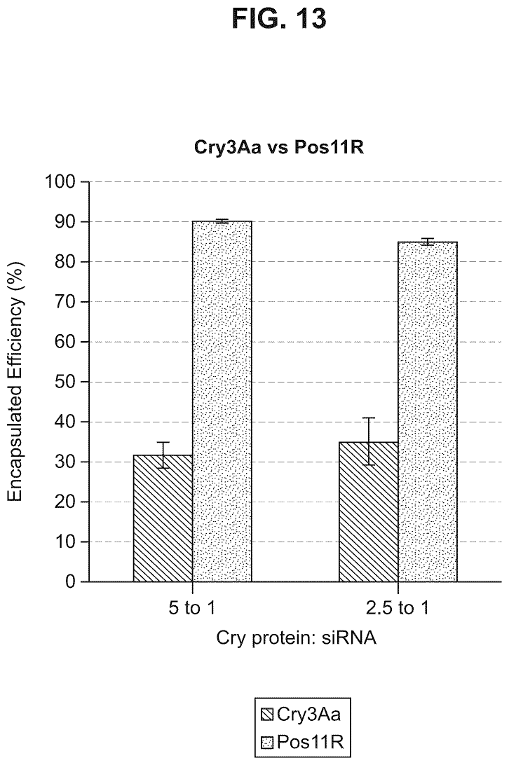

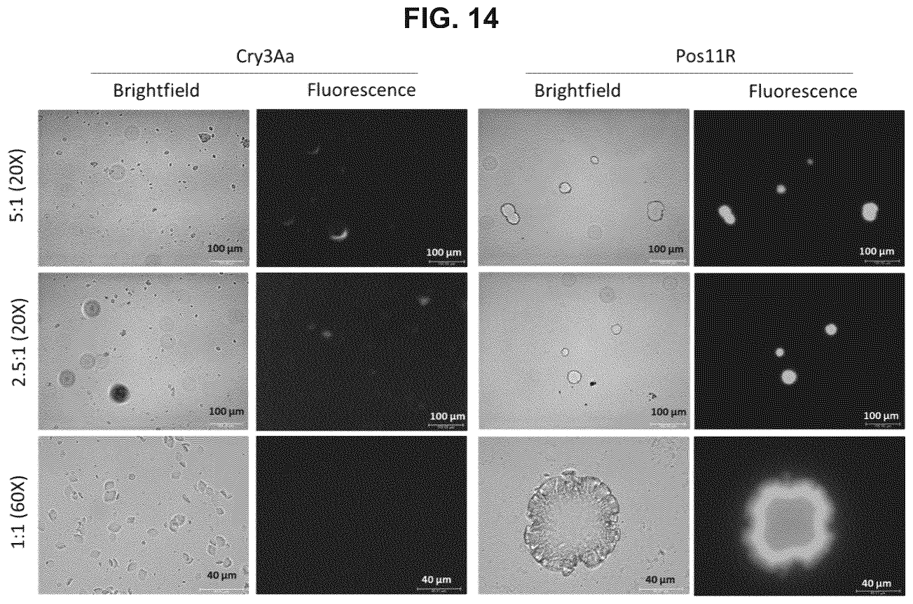

After significant optimization of the recrystallization conditions, surprisingly high siRNA loadings were able to be achieved, and it was determined that siRNA size up to 100 bp did not reduce the encapsulation efficiency. It was also important to distinguish whether the positively charged mutations within Pos11R were responsible for the observed high encapsulation efficiencies and loadings. As such, Cry3Aa wild-type and Pos11R at 5:1 and 2.5:1 crystal protein:siRNA ratios were compared. The encapsulation efficiencies of Pos11R were significantly higher than Cry3Aa wild-type at both ratios tested (FIG. 13), indicating the mutations within Pos11R are responsible for the high siRNA loading.

Examined next were the Pos11R and Cry3Aa crystals after encapsulation of FAM-siRNA at different crystal protein:siRNA molar ratios under a fluorescent microscope (FIG. 14). Cry3Aa formed tear-drop shaped crystals of ˜20 μm in size at all molar ratios examined. This shape is consistent with the SEM data presented in FIG. 8. However, the Cry3Aa crystals did exhibit noticeable fluorescence, which is consistent with their low encapsulation efficiencies observed in FIG. 13. On the other hand, Pos11R crystals exhibited bright fluorescence under all Pos11R: siRNA molar ratios examined, indicating large amounts of FAM-siRNA were present in the crystals. The shape of the Pos11R crystals were more circular, which is similar to their shape when produced in Bt. However, their sizes range from 20-40 μm, which were slightly larger than wild-type Cry3Aa. This fluorescence microscopy data further corroborates that the Pos11R crystals contain FAM-siRNA within the crystals.

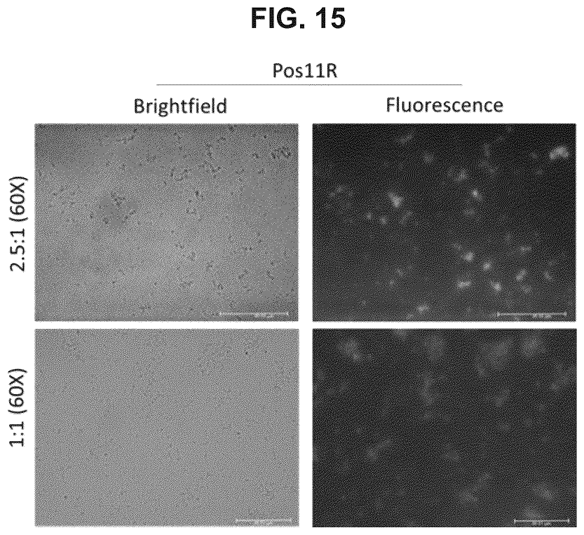

Upon further examination of the microscopy data for the Pos11R crystals post-encapsulation, observed were very small particles that were also fluorescent, suggesting they contained FAM-siRNA (FIG. 15). This observation suggested that there may be two different sized crystals forming.

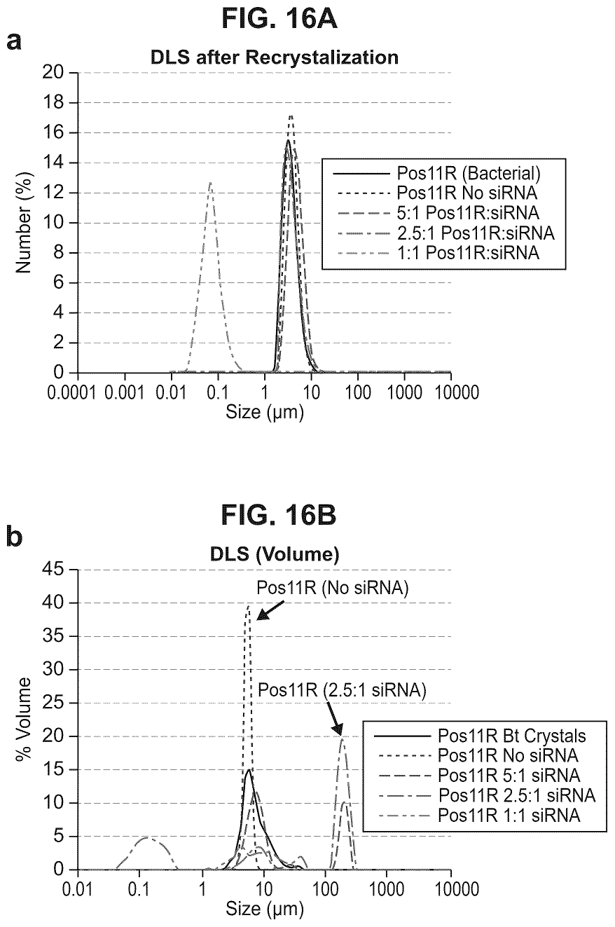

In light of the possibility of the formation of two different crystal forms post-recrystallization, dynamic light scattering (DLS) was employed to determine the crystal sizes within the population. When examining number of particles, Pos11R crystals produced in bacteria, Pos11R crystals recrystallized in the absence of siRNA, and Pos11R crystals recrystallized in the presence of siRNA at 2.5:1 and 1:1 Pos11R:siRNA ratios had similar sizes (FIG. 16a). Interestingly, the particle size of Pos11R crystals recrystallized at a 1:1 Pos11R:siRNA ratio was significantly smaller and in the nm range. This result was unexpected, but this size range could be small enough to penetrate the plant cuticle, enabling delivery of nucleic acids (siRNAs, etc.) directly to plants to protect against viruses and fungi.

When evaluating % volume distribution, some significant differences were observed (FIG. 16b). It was discovered that altering the Pos11R:siRNA ratio significantly changes the particle size distribution. Without siRNA, a sharp peak of crystals with a similar size distribution is observed. At a 5:1 Pos11R:siRNA ratio there is about a 50/50 mix of small and large particles, and this shifts to mostly larger particles at a 2.5:1 Pos11R:siRNA ratio. Surprisingly, a significant shift in the opposite direction was observed at a 1:1 ratio. This may be due to the crystal proteins being saturated with siRNA bound to them, which may disrupt further nucleation. The DLS (volume) data agrees with the microscopy data, where two different particle sizes were observed.

### Example 3—Stability of Encapsulated Negatively-Charged Biomolecules

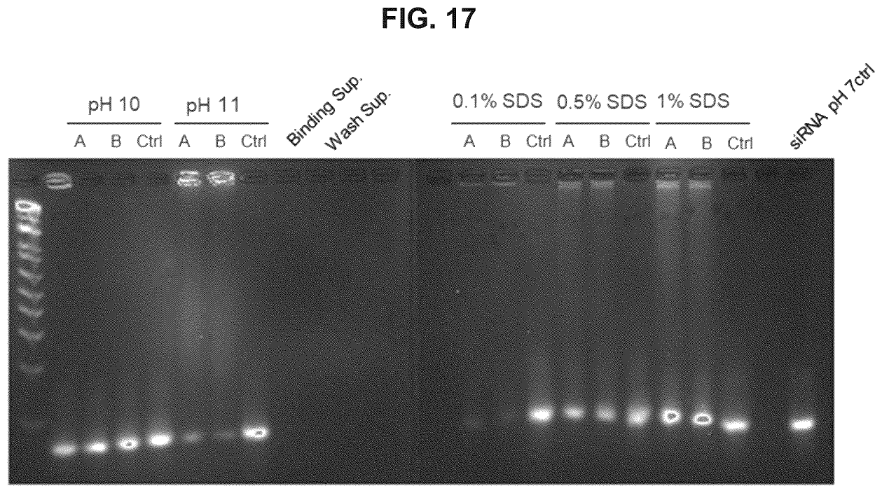

A crucial milestone to the development of a viable product is to demonstrate stability of the negatively-charged biomolecule in the crystal. siRNA was here again used as proof of concept. An assay was first to probe the stability of siRNA by releasing it from the crystal and implementing gel electrophoresis and subsequent siRNA staining by ethidium bromide to detect RNA degradation. Prior to testing this RNA degradation assay, several conditions were screened for release of the siRNA following direct binding (FIG. 17). Tested were two different pH buffers, pH 10 and pH 11, which are known to solubilize the crystals, as well as varying concentrations of sodium dodecyl sulfate (SDS), which can dissolve protein crystals yet will not perturb the RNA. Based on the data, all conditions were sufficient to release the siRNA, except 0.1% SDS. pH 10 was selected for further investigations since nearly all siRNA was released, and this condition is known to solubilize the crystals without denaturing the protein.

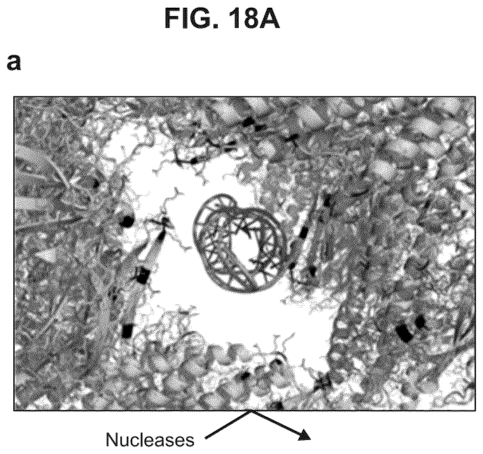

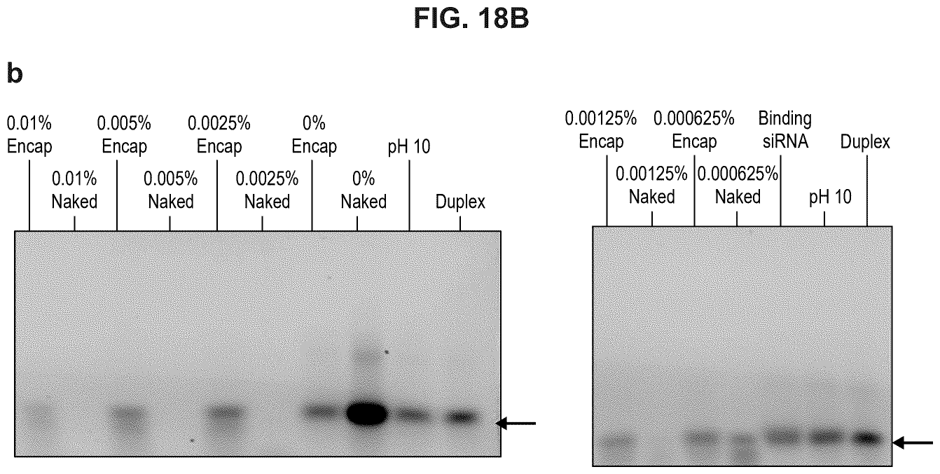

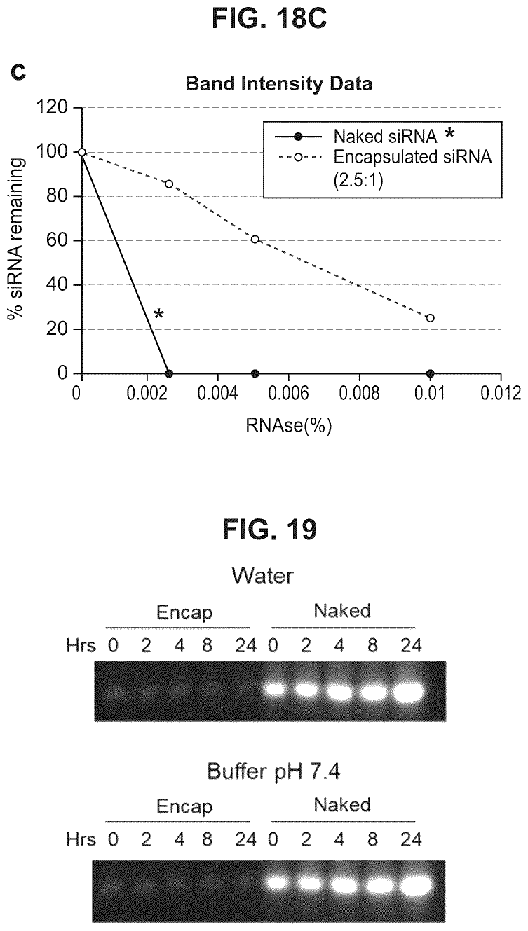

Assessed next was whether the siRNA is shielded (i.e., within the solvent channels inside the crystal) or simply binding to the surface, since shielding within the crystal will offer better protection from the environment (e.g., digestive enzymes in the insect midgut and from ultraviolet light), resulting in a more stable product. To address this question, encapsulated siRNA was exposed to an RNAse enzyme, which will degrade the siRNA bound to the surface but cannot enzymatically cleave siRNAs within the channels, since they are inaccessible (FIG. 18a). This is a common assay to quickly measure siRNA stability, since RNAse will rapidly degrade siRNA within minutes. The Pos11R crystals were incubated with siRNA to encapsulate it by recrystallization, and then these Pos11R-siRNA crystals and naked siRNA were incubated in various concentrations of RNAse (% v/v). Then, the siRNA was released from the crystal at pH 10 and all samples were loaded onto an agarose gel as before to detect degradation products. The data in FIG. 18b shows that naked siRNA begins to degrade at 0.000625% RNAse after a 30 min incubation, and nearly all naked siRNA is degraded at 0.00125% RNAse. On the other hand, encapsulated siRNA could still be observed at the highest concentration of RNAse used (0.01% v/v). Densitometry analysis was used to quantitate the band intensities and is shown in FIG. 18C. This data not only demonstrates that siRNA is more stable than naked siRNA following encapsulation, but it also supports that the siRNA is encapsulated within the crystal.

Finally, compared was the long-term stability of encapsulated siRNA to naked siRNA in solution. First, siRNA encapsulated by recrystallization and naked siRNA at the same concentration were incubated in a buffer at physiological pH and in tap water, which acts as a rain simulant, and timepoints were taken up to 24 hours (FIG. 19). There was not an obvious decrease in band intensity from 0 hours to 24 hours in either condition, suggesting both siRNAs are stable for 24 hrs. A large difference in band intensity was observed between naked and encapsulated (Encap) siRNA on the agarose gel. The data in FIG. 12a confirms that all loaded siRNA is released by incubating at pH 10. The other major difference between the prepared Encap sample and naked sample is that the siRNA is first dissolved in citrate buffer, then mixed with the protein sample, while naked siRNA was diluted directly in the target solution. This buffer change may explain the difference in band intensity, since lower pH is known to alter the binding ethidium bromide to the RNA when staining.

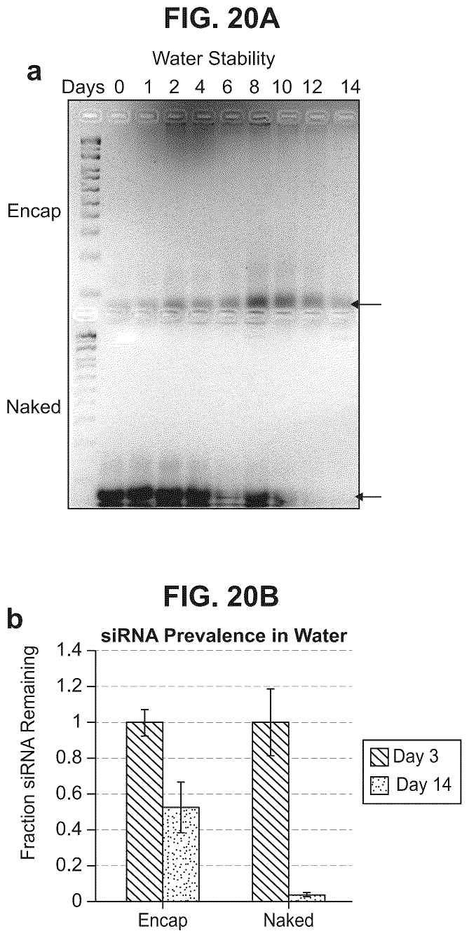

Given that siRNA was stable in tap water for 24 hours, the next experiment was performed for two weeks in tap water, and samples were taken and evaluated for degradation using the agarose gel assy. Again, a difference in band intensity was observed between the naked and encapsulated siRNA. An observable band could still be seen after 14 days with the encapsulated sample, but the naked siRNA band nearly fully disappeared (FIG. 20a). When these bands were quantified for intensity, the data demonstrated that the siRNA was more than a log-fold more stable when encapsulated using the Cry encapsulation platform compared to naked siRNA (FIG. 20b). These data highlight the potential of the Cry platform to stabilize the siRNA in the environment.

In conclusion, these proof-of-concept results illustrate the feasibility of this approach for encapsulating negatively-charged biomolecules, including but not limited to nucleic acids such as siRNA, etc. The loading of siRNA was significantly improved compared to the initial recrystallization test, and an 11-fold improvement in siRNA loading was achieved, while still retaining high encapsulation efficiency compared to the direct encapsulation approach. 40% w/w was achieved by using optimized conditions for recrystallization, and stimuli-responsive release of siRNA from the crystal in response to a simulated midgut pH was also observed. Also demonstrated was protection of the siRNA after encapsulation, both from nuclease-dependent degradation and long-term stability in tap water. There are several differentiators of this approach that set this technology apart from others, including a very high loading of the negatively-charged biomolecule (siRNA in this example), an inherent release mechanism for the negatively-charged biomolecule at the site of action, the potential of synergism between the Cry protein and siRNA, which would lead to increased efficacy and decreased chance of gaining resistance, and that Cry proteins have been used in the industry for several decades, such that their large scale formulation, production, dissemination, toxicity profile, and economics are already understood and well established.

### Methods

Cloning of Cry3Aa Variants into the pHT315 Expression Vector

The cry3Aa gene was modified to generate a super positive mutant (SupPosA), which had 14 amino acids changed to arginine to allow for a higher positive charge within the binding channel of the protein. The SupPosA mutant was built off of to produce genes that when translated into proteins were slightly less charged and had a total of 5 arginine (5R), 8 arginine (8R) or 11 arginine (11R).

These new variants were all cloned into the pHT315 plasmid that was constructed for the expression of the SupPosA variant. The SupPosA plasmid was cut using restriction digestion and a gblock of the mutated sequence was inserted into the plasmid via Gibson assembly using the NEB HiFi DNA assembly mix kit with a 1:2 vector: insert ratio. (Appendix 1).

The new pHT315 expression vectors harboring the Pos5R, Pos8R and Pos11R genes were transformed in DH5alpha E. coli cells and sequenced to confirm correct insertion and gene sequence. Prior to transformation in Bacillus thuringiensis 4A12 cells, plasmids were transformed into expressed SCS110 E. coli cells, which demethylates the plasmids.

### S145C Mutant of Pos11R

To confirm that the Pos11R crystals formed the same lattice structure as wild-type Cry3Aa, a mutant of Pos11R was generated to convert serine 145 into a cysteine to allow for disulfide bonding. The S145C mutant was achieved by designing primers that would mutate the site from the Pos11R template. The mutagenesis PCR was conducted using the Kapa HiFi HotStart ready mix and a gradient of annealing temperature from 63-69° C.; with 63° C. and 66° C. being the most effective. The product was then DpnI digested and transformed into DH5alpha E. coli cells. A selection of the resulting colonies was sequenced to confirm the mutation and subsequently transformed into SCS110 for demethylation.

Bacillus thuringiensis Transformations

Bacillus thuringiensis 4A12 competent cells were made before the transformation and were used the same day they were made. 1 μg of each corresponding plasmid (Pos5R, Pos8R, Pos11R, shRNA T4, and S145C) was added to the Bacillus thuringiensis 4A12 competent cells. The cells then underwent electroporation at 2.5 kV in Biorad Gene Pulser, 0.2 cm cuvettes. BHI broth was spiked with the electroporated cells and then incubated for 4 hours at 30° C. with 250 RPM. Post incubation, 50 μL of the 4-hour culture was plated on LB agar with 50 μg of erythromycin. Plates were incubated overnight at 30° C.

### Crystal Production

All strains of Bt4A12 were inoculated into Schaefer's Sporulation Medium (SSM) with 50 μg/mL erythromycin. Cells were incubated for 72 hours at 25° C. with 220 RPM. The crystal-spore culture was centrifuged at 8,000 RPM for 7 minutes. The pellet was washed with sterile DI water and then resuspended in 20 mL of sterile DI water. 200 μL of 100 mg/mL Lysozyme was added to resuspended pellet and incubated overnight at room temperature. The next day sonication was used to complete cell lysis. Sonication was run at 60% amplification for a total of 10 minutes with cycles of 10 seconds on and 10 seconds off. This was repeated till 100% cell lysis was observed under a phase contrast microscope. The lysed sample was centrifuged at 8,500 RPM for 7 minutes and then the supernatant was discarded. A series of two 1M NaCl washes and two sterile DI washes were used on the sample after centrifugation. The sample was then once again centrifuged at 8,500 RPM and the supernatant was discarded, while the pellet was resuspended in 5 mL of sterile DI water and stored at 4° C.

siRNA Synthesis

RNA oligonucleotides FAM-DvSnf7 Forward, DvSnf7 Forward, and DvSnf7 Reverse were purchased from IDT and then dissolved in nuclease free water to make 100 μM stocks. FAM-DvSnf7 siRNA and DvSnf7 siRNA were produced by mixing DvSnf7 Reverse with either FAM-DvSnf7 Forward or DvSnf7 Forward in a 1:1 ratio. These mixtures were then heated to 75° C. in a thermocycler or thermomixer for 2 minutes. Mixtures were cooled at room temperature for 10 minutes and then promptly incubated on ice for 10 minutes. These samples were run on gel electrophoresis to identify if the siRNA was synthesized.

### Solubilization

Bacillus thuringiensis 4A12 variants Pos5R, Pos8R, Pos11R, supPos3a and the control strain Cry3A underwent the same solubilization method. The protein crystal was spun down in a centrifuge at 8,000 RPM for 5 minutes. The supernatant was removed and then pH 11, 0.1 M NaCO₃ was added so the protein crystal concentration would be 1 mg/mL. The protein crystal pellet was then resuspended and incubated overnight at room temperature at 750 RPM. The next day, the solubilized crystal would be spun down at 8,500 RPM for 15 minutes. The supernatant would be filtered using a 0.45 μm syringe filter. The concentration was then measured via Bradford assay. The spectrophotometer was set to a wavelength of 595 nm.

### DLS Prep

Protein crystals from variant Pos11R and recrystallized Pos11R were analyzed via DLS. The Pos11R protein crystals were aliquoted from a stock so the concentration would be 0.2 mg/mL. This aliquot was centrifuged for 5 minutes at 13,000 RPM and then the supernatant discarded. Solubilized Pos11R protein crystals were diluted to 1 mg/mL. siRNA stocks were created at concentrations of 0.55, 1.1, and 2.74 UM with 50 UM FAM-DvSnf7 siRNA and pH 4.0, 0.1 M Citrate buffer. The siRNA stocks and solubilized protein crystal were combined at a 4:1 ratio (100 μL crystal to 400 μL siRNA stock). These mixtures were incubated for 1 hour at 25° C. with 750 RPM. These recrystallized samples were then centrifuged. 150 μL of supernatant was kept for fluorescent spectroscopy and the rest was discarded. The Pos11R protein crystal and the recrystallized Pos11R were washed three times with nuclease free water. The pellets were then resuspended in nuclease free water and stored at 4° C.

### Optimal Recrystallization Approach

Protein crystals were used previously solubilized utilizing the solubilization method. Their concentrations were adjusted to 1 mg/mL. Stocks of FAM-DvSnf7 siRNA were made at the concentrations 0.55 μM, 1.10 UM, and 2.74 μM from 50 μM FAM-DvSnf7 siRNA and 0.1M Citrate Buffer pH 4. The stocks were added in a 4:1 ratio of siRNA stock to solubilized crystal. These were incubated for 30 minutes at 25° C. with 750 RPM. The recrystallized mixture was then centrifuged for 5 minutes at 13,000 RPM. Supernatants were aliquoted and diluted based on siRNA to solubilized protein concentration. The 0.55 μM supernatant had 50 μL of sample aliquoted to a 96-well plate and had 50 μL of duplex added to the well. The 1.10 UM supernatant had 25 μL aliquoted to a 96-well plate and 75 μL of duplex added as well. The 2.74 μM supernatant had 10 μL aliquoted to a 96-well plate and then 90 μL of duplex added. The remaining supernatant was discarded and the pellets were washed with 20% duplex buffer and then nuclease free water. The remaining supernatant was discarded the pellets were stored at 4° C. until they were imaged under a LEICA microscope.

Recrystallization Encapsulation of Long siRNA

Three different lengths of DvSnf7 siRNA (DvSnf7 60, 80, and 100) were generated using the siRNA synthesis method. These siRNA were prepared into stocks at a concentration of 1.375 μM in 0.1 M citrate buffer pH 4. The stocks were then added at a 4:1 ratio of siRNA stock to Pos11R solubilized protein. This mixture was incubated for 30 minutes at 25° C. with 750 RPM. Samples were then centrifuged for 5 minutes at 13,000 RPM and the supernatant was discarded. The pellet was washed once with 20% duplex buffer and once with nuclease free water. The washed pellets were resuspended in nuclease free water and then ran on a 4% agarose gel and on a spectrophotometer. The agarose gel ran for 80 minutes at 120 V and imaged on an EZ Gel Doc Imager. The spectrophotometer was set to an excitation of 493 nm and an emission of 519 nm.

Stability of siRNA

Stability of siRNA was assessed across various time points and concentrations of RNase. Pos11R protein crystals were solubilized following the Recrystallization Encapsulation methods with a siRNA to protein crystal ratio of 5:1, 2.5, and 1:1. The recrystallized crystal was then centrifuged for 5 minutes at 13,000 RPM and the supernatant discarded. This pellet was washed with 20% duplex buffer and then nuclease free water. RNase stocks were made at the following RNase percentages: 0.01%, 0.005%, 0.0025%, 0.00125%, and 0.000625% RNase. These stocks were used to resuspend to the recrystallized pellet and incubated for 1 hour at 25° C. with 500 RPM. Samples were then spun down in a centrifuge for 5 minutes at 13,000 RPM and the supernatant discarded. The remaining pellet was washed with nuclease free water and then resuspended with 0.1 M Na₂CO₃ pH 10. The resuspended pellet is incubated for 1 hour at 25° C. with 500 RPM. Solubilized protein was centrifuged, the supernatant collected and then ran on a 4% agarose gel with ethidium bromide for 80 minutes at 120 V. Gels were imaged on EZ Gel Doc Imager.

Time-based stability assays used the same method with the exception that aliquots were taken at each time point. This aliquot was then centrifuged for 5 minutes, and the supernatant was discarded. The pellet was then washed with nuclease free water. The pellet was then resuspended in 0.1 M Na₂CO₃ pH 10 and then incubated for 1 hour at 25° C. with 500 RPM. Solubilized protein was centrifuged and then supernatant was collected. Supernatant had 6×DNA loading dye added and then samples were stored at −20° C. until all time points were pulled and processed. Samples were run on a 4% agarose gel with ethidium bromide for 80 minutes at 120 V. Gels were imaged on EZ Gel Doc Imager.

### RNA Oligo Sequences

27mer that Targets DvSnf7 is Underlined

(SEQ ID NO: 11)
DvSnf7 27mer in 60 bp Fwd
UUUUGUAGAUUAAGUGCUAGAUGGAACCCUUACAACUAUUGAAAA
UCUAAGAUAGGAAGG
DvSnf7 27mer in 60 bp Rev
(SEQ ID NO: 12)
CCUUCCUAUCUUAGAUUUUCAAUAGUUGUAAGGGUUCCAUCUAGC
ACUUAAUCUACAAAA
DvSnf7 27mer in 80 bp Fwd
10
(SEQ ID NO: 13)
AGUGAGAUACUUUUGUAGAUUAAGUGCUAGAUGGAACCCUUACAA
CUAUUGAAAAUCUAAGAUAGGAAGGCGAGAAUGAA
DvSnf7 27mer in 80 bp Rev
(SEQ ID NO: 14)
UUCAUUCUCGCCUUCCUAUCUUAGAUUUUCAAUAGUUGUAAGGGU
UCCAUCUAGCACUUAAUCUACAAAAGUAUCUCACU
15
DvSnf7 27mer in 100 bp Fwd
(SEQ ID NO: 15)
UCAGUGCCUGAGUGAGAUACUUUUGUAGAUUAAGUGCUAGAUGGA
ACCCUUACAACUAUUGAAAAUCUAAGAUAGGAAGGCGAGAAUGAA
UUAUGGAAAU
DvSnf7 27mer in 100 bp Rev
(SEQ ID NO: 16)
AUUUCCAUAAUUCAUUCUCGCCUUCCUAUCUUAGAUUUUCAAUAG
UUGUAAGGGUUCCAUCUAGCACUUAAUCUACAAAAGUAUCUCACU
CAGGCACUGA

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1.-75. (canceled)

76. A Cry3Aa protein crystal comprising a negatively charged biomolecule encapsulated therein, wherein the solvent channel of the Cry3Aa protein crystal comprises an amino acid substitution of an uncharged or negatively charged amino acid for a positively charged amino acid, and wherein encapsulation of the biomolecule comprises interaction of the biomolecule with the positively charged amino acid.

77. The Cry3Aa protein crystal of claim 76, wherein the amino acid substitution comprises substitution of an uncharged or negatively charged amino acid for an arginine (R) or lysine (K).

78. The Cry3Aa protein crystal of claim 76, wherein the uncharged or negatively charged amino acid is an aspartic acid (D), glutamic acid (E), serine(S), asparagine (N), or glutamine (Q).

79. The Cry3Aa protein crystal of claim 76, wherein the Cry3Aa protein crystal comprises Cry3Aa proteins comprising an amino acid substitution chosen from D118R, S187R, S257R, S322R, N326R, N391R, N395R, E423R, Q430R, D432R, E433R, E466R, A629R, or any combination thereof, and wherein numbering is as in SEQ ID NO: 1.

80. The Cry3Aa protein crystal of claim 79, wherein the Cry3Aa proteins comprise from 5 to 11 of the substitutions.

81. The Cry3Aa protein crystal of claim 76, wherein the solubility of the Cry3Aa protein crystal is greater than 60% of the solubility of a wild-type Cry3A protein crystal, wherein solubility is determined in a 15 mL solubilization reaction at pH 11 and a crystal concentration of 1 mg/ml.

82. The Cry3Aa protein crystal of claim 76, wherein the negatively charged biomolecule is a nucleic acid.

83. The Cry3Aa protein crystal of claim 82, wherein the nucleic acid is adapted to reduce expression of a target gene by RNA interference (RNAi).

84. The Cry3Aa protein crystal of claim 83, wherein the nucleic acid is a short interfering RNA (siRNA), a short hairpin RNA (shRNA), a DROSHA substrate, a Dicer substrate, a microRNA (miRNA), or a PIWI-interacting RNA (piRNA).

85. The Cry3Aa protein crystal of claim 82, wherein the nucleic acid interferes with expression of a gene of a pest, a bacterium, a virus, a fungus, or a parasite.

86. The Cry3Aa protein crystal of claim 85, wherein the pest is an agricultural pest.

87. A composition comprising a plurality of Cry3Aa protein crystals of claim 76.

88. A method of controlling a pest, a bacterium, a virus, a fungus, or a parasite, the method comprising delivering an effective amount of the composition of claim 87 to the pest, bacterium, virus, fungus, or parasite.

89. The method of claim 88, wherein the delivering comprises applying an effective amount of the composition to a crop plant, optionally wherein the crop plant is rice, maize, sugarcane, wheat, barley, soybean, potato, tomato, cucumber, lettuce, onion, cotton, rapeseed, coffee, tea, tobacco, or hops.

90. A nucleic acid encoding a Cry3Aa protein of the Cry3Aa protein crystal of claim 76.

91. A cell comprising an expression construct comprising the nucleic acid of claim 90 operably linked to a promoter.

92. The cell of claim 91, wherein the cell is a prokaryotic cell.

93. The cell of claim 92, wherein the prokaryotic cell is a Bacillus thuringiensis (Bt) cell.

94. A method of producing Cry3Aa protein crystals comprising a negatively charged biomolecule encapsulated therein, the method comprising:
(a) incubating the cell of claim 92 under conditions in which Cry3Aa protein crystals are produced, and
(b) encapsulating a negatively charged biomolecule in the Cry3Aa protein crystals; or
(a) solubilizing Cry3Aa protein crystals to produce solubilized Cry3Aa protein, and
(b) recrystallizing the solubilized Cry3Aa protein in the presence of the negatively charged biomolecule.

95. The method of claim 94, wherein the method comprises:
(a) incubating the cell under conditions in which Cry3Aa protein crystals are produced, and
(b) encapsulating a negatively charged biomolecule in the Cry3Aa protein crystals,
wherein encapsulating the negatively charged biomolecule comprises:
isolating the Cry3Aa protein crystals produced during the incubating step;
solubilizing the isolated Cry3Aa protein crystals to produce solubilized Cry3Aa protein; and
recrystallizing the solubilized Cry3Aa protein in the presence of the negatively charged biomolecule,
wherein solubilizing the isolated Cry3Aa protein crystals comprises incubating the isolated Cry3Aa protein crystals in an alkaline buffer,
wherein recrystallizing the solubilized Cry3Aa protein in the presence of the negatively charged biomolecule comprises adding an acidic buffer comprising the negatively charged biomolecule to the alkaline buffer comprising the solubilized Cry3Aa protein, and
wherein the volume of added acidic buffer provides a ratio of acidic buffer to alkaline buffer of from 3:1 to 5:1 or from 3.5:1 to 4.5:1, optionally about 4:1.


SEQUENCE LISTING (16 sequences)

SEQ ID NO: 1 - protein, 644 aa, Bacillus thuringiensis
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 2 - DNA, 1935 bp, Bacillus thuringiensis
  source 1..1935: mol_type = genomic DNA
atgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatggatcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaatttctggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggttcatcttatgaatcttgggtaaactttaaccgttatcgcagagagatgacattaacagtattagatttaattgcactatttccattgtatgatgttcggctatacccaaaagaagttaaaaccgaattaacaagagacgttttaacagatccaattgtcggagtcaacaaccttaggggctatggaacaaccttctctaatatagaaaattatattcgaaaaccacatctatttgactatctgcatagaattcaatttcacacgcggttccaaccaggatattatggaaatgactctttcaattattggtccggtaattatgtttcaactagaccaagcataggatcaaatgatataatcacatctccattctatggaaataaatccagtgaacctgtacaaaatttagaatttaatggagaaaaagtctatagagccgtagcaaatacaaatcttgcggtctggccgtccgctgtatattcaggtgttacaaaagtggaatttagccaatataatgatcaaacagatgaagcaagtacacaaacgtacgactcaaaaagaaatgttggcgcggtcagctgggattctatcgatcaattgcctccagaaacaacagatgaacctctagaaaagggatatagccatcaactcaattatgtaatgtgctttttaatgcagggtagtagaggaacaatcccagtgttaacttggacacataaaagtgtagacttttttaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtgctggagataaagtttatatagacaaaattgaatttattccagtgaattaa

SEQ ID NO: 3 - protein, 644 aa, synthetic construct
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMRQKIADYAKNKALAELQGLQNNVEDYVSALSSWQKNPVSSRNPHSQGRIRELFSQAESHFRNSMPSFAIRGYEVLFLTTYAQAANTHLFLLKDAQIYGEEWGYEKEDIAEFYKRQLKLTQEYTDHCVKWYNVGLDKLRGRSYESWVNFNRYRREMTLTVLDLIALFPLYDVRLYPKEVKTELTRDVLTDPIVGVNNLRGYGTTFSNIERYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVQFSQYNDQTNQASTQTYDSKRNVGAVSWDSIDQLPPETTDEPLQKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSRGDKVYIDKIEFIPVN

SEQ ID NO: 4 - DNA, 1935 bp, synthetic construct
  source 1..1935: mol_type = other DNA
atgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatgcgtcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaattcgtggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggtagatcttatgaatcttgggtaaactttaaccgttatcgcagagagatgacattaacagtattagatttaattgcactatttccattgtatgatgttcggctatatccgaaagaagttaaaacggaattgacaagagacgttcttacagatccaatcgtcggtgttaataacctccggggttatgggacgacattcagcaatatcgagagatacatacgtaaacctcacttgtttgactatttacatcgcatacagttccatacccggttccaacctggatactacggaaacgactcttttaactactggtctggtaattacgtcagcacccggcctagcataggctcaaacgatattatcacgagccctttctacggaaataaaagctcagagccagtacaaaacttagaattcaacggtgaaaaggtgtatagagccgttgcgaataccaacttagcggtatggccgagtgccgtatattctggtgtaacaaaggtacagtttagccaatataacgatcagacgaaccaggcgtccacccaaacatacgacagcaaacgtaacgttggagcagtttcatgggattctatagatcaacttccgccagaaacaacagatgaaccacttcagaagggttattcacaccagctcaattacgtgatgtgttttctgatgcagggatcacgcgggaccattccagtcttgacctggacgcataaaagtgtagactttttcaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtcgtggagataaagtttatatagacaaaattgaatttattccagtgaattaa

SEQ ID NO: 5 - protein, 644 aa, synthetic construct
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMRQKIADYAKNKALAELQGLQNNVEDYVSALSSWQKNPVSSRNPHSQGRIRELFSQAESHFRNSMPSFAIRGYEVLFLTTYAQAANTHLFLLKDAQIYGEEWGYEKEDIAEFYKRQLKLTQEYTDHCVKWYNVGLDKLRGRSYESWVNFNRYRREMTLTVLDLIALFPLYDVRLYPKEVKTELTRDVLTDPIVGVNNLRGYGTTFSNIERYIRKPHLFDYLHRIQFHTRFQPGYYGNDSFNYWSGNYVSTRPSIGSNDIITSPFYGNKSSEPVQRLEFNGEKVYRAVANTNLAVWPSAVYSGVTKVQFSQYNDQTNRASTQTYDSKRNVGAVSWDSIDQLPPETTDEPLRKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSRGDKVYIDKIEFIPVN

SEQ ID NO: 6 - DNA, 1935 bp, synthetic construct
  source 1..1935: mol_type = other DNA
atgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatgcgtcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaattcgtggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggtagatcttatgaatcttgggtaaactttaaccgttatcgcagagagatgacattaacagtattagatttaattgcactatttccattgtatgatgttcggctatatccgaaagaagttaaaacggaattgacaagagacgttcttacagatccaatcgtcggtgttaataacctccggggttatgggacgacattcagcaatatcgagagatacatacgtaaacctcacttgtttgactatttacatcgcatacagttccatacccggttccaacctggatactacggaaacgactcttttaactactggtctggtaattacgtcagcacccggcctagcataggctcaaacgatattatcacgagccctttctacggaaataaaagctcagagccagtacaaagattagaattcaacggtgaaaaggtgtatagagccgttgcgaataccaacttagcggtatggccgagtgccgtatattctggtgtaacaaaggtacagtttagccaatataacgatcagacgaacagagcgtccacccaaacatacgacagcaaacgtaacgttggagcagtttcatgggattctatagatcaacttccgccagaaacaacagatgaaccacttagaaagggttattcacaccagctcaattacgtgatgtgttttctgatgcagggatcacgcgggaccattccagtcttgacctggacgcataaaagtgtagactttttcaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtcgtggagataaagtttatatagacaaaattgaatttattccagtgaattaa

SEQ ID NO: 7 - protein, 644 aa, synthetic construct
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMRQKIADYAKNKALAELQGLQNNVEDYVSALSSWQKNPVSSRNPHSQGRIRELFSQAESHFRNSMPSFAIRGYEVLFLTTYAQAANTHLFLLKDAQIYGEEWGYEKEDIAEFYKRQLKLTQEYTDHCVKWYNVGLDKLRGRSYESWVNFNRYRREMTLTVLDLIALFPLYDVRLYPKEVKTELTRDVLTDPIVGVNNLRGYGTTFSNIERYIRKPHLFDYLHRIQFHTRFQPGYYGNDSFNYWSGNYVSTRPSIGSNDIITSPFYGNKSSEPVQRLEFNGEKVYRAVANTNLAVWPSAVYSGVTKVRFSQYNDRTNRASRQTYDSKRNVGAVSWDSIDQLPPETTDEPLRKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSRGDKVYIDKIEFIPVN

SEQ ID NO: 8 - DNA, 1935 bp, synthetic construct
  source 1..1935: mol_type = other DNA
atgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatgcgtcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaattcgtggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggtagatcttatgaatcttgggtaaactttaaccgttatcgcagagagatgacattaacagtattagatttaattgcactatttccattgtatgatgttcggctatatccgaaagaagttaaaacggaattgacaagagacgttcttacagatccaatcgtcggtgttaataacctccggggttatgggacgacattcagcaatatcgagagatacatacgtaaacctcacttgtttgactatttacatcgcatacagttccatacccggttccaacctggatactacggaaacgactcttttaactactggtctggtaattacgtcagcacccggcctagcataggctcaaacgatattatcacgagccctttctacggaaataaaagctcagagccagtacaaagattagaattcaacggtgaaaaggtgtatagagccgttgcgaataccaacttagcggtatggccgagtgccgtatattctggtgtaacaaaggtacgttttagccaatataacgatcgtacgaacagagcgtcccgtcaaacatacgacagcaaacgtaacgttggagcagtttcatgggattctatagatcaacttccgccagaaacaacagatgaaccacttagaaagggttattcacaccagctcaattacgtgatgtgttttctgatgcagggatcacgcgggaccattccagtcttgacctggacgcataaaagtgtagactttttcaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtcgtggagataaagtttatatagacaaaattgaatttattccagtgaattaa

SEQ ID NO: 9 - protein, 644 aa, synthetic construct
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMRQKIADYAKNKALAELQGLQNNVEDYVSALSSWQKNPVSSRNPHSQGRIRELFSQAESHFRNSMPSFAIRGYEVLFLTTYAQAANTHLFLLKDAQIYGEEWGYEKEDIAEFYKRQLKLTQEYTDHCVKWYNVGLDKLRGRSYESWVNFNRYRREMTLTVLDLIALFPLYDVRLYPKEVKTELTRDVLTDPIVGVNNLRGYGTTFRNIERYIRKPHLFDYLHRIQFHTRFQPGYYGNDSFNYWSGNYVSTRPSIGSNDIITSPFYGNKSSEPVQRLEFRGEKVYRAVANTNLAVWPSAVYSGVTKVRFSQYNDRTRRASRQTYDSKRNVGAVSWDSIDQLPPETTDEPLRKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSRGDKVYIDKIEFIPVN

SEQ ID NO: 10 - DNA, 1935 bp, synthetic construct
  source 1..1935: mol_type = other DNA
atgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatgcgtcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaattcgtggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggtagatcttatgaatcttgggtaaactttaaccgttatcgcagagagatgacattaacagtattagatttaattgcactatttccattgtatgatgttcggctatatccgaaagaagttaaaacggaattgacaagagacgttcttacagatccaatcgtcggtgttaataacctccggggttatgggacgacattcagaaatatcgagagatacatacgtaaacctcacttgtttgactatttacatcgcatacagttccatacccggttccaacctggatactacggaaacgactcttttaactactggtctggtaattacgtcagcacccggcctagcataggctcaaacgatattatcacgagccctttctacggaaataaaagctcagagccagtacaaagattagaattcagaggtgaaaaggtgtatagagccgttgcgaataccaacttagcggtatggccgagtgccgtatattctggtgtaacaaaggtaagatttagccaatataacgatagaacgagaagagcgtccagacaaacatacgacagcaaacgtaacgttggagcagtttcatgggattctatagatcaacttccgccagaaacaacagatgaaccacttagaaagggttattcacaccagctcaattacgtgatgtgttttctgatgcagggatcacgcgggaccattccagtcttgacctggacgcataaaagtgtagactttttcaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtcgtggagataaagtttatatagacaaaattgaatttattccagtgaattaa

SEQ ID NO: 11 - RNA, 60 nt, synthetic construct
  source 1..60: mol_type = other RNA
ttttgtagattaagtgctagatggaacccttacaactattgaaaatctaagataggaagg

SEQ ID NO: 12 - RNA, 60 nt, synthetic construct
  source 1..60: mol_type = other RNA
ccttcctatcttagattttcaatagttgtaagggttccatctagcacttaatctacaaaa

SEQ ID NO: 13 - RNA, 80 nt, synthetic construct
  source 1..80: mol_type = other RNA
agtgagatacttttgtagattaagtgctagatggaacccttacaactattgaaaatctaagataggaaggcgagaatgaa

SEQ ID NO: 14 - RNA, 80 nt, synthetic construct
  source 1..80: mol_type = other RNA
ttcattctcgccttcctatcttagattttcaatagttgtaagggttccatctagcacttaatctacaaaagtatctcact

SEQ ID NO: 15 - RNA, 100 nt, synthetic construct
  source 1..100: mol_type = other RNA
tcagtgcctgagtgagatacttttgtagattaagtgctagatggaacccttacaactattgaaaatctaagataggaaggcgagaatgaattatggaaat

SEQ ID NO: 16 - RNA, 100 nt, synthetic construct
  source 1..100: mol_type = other RNA
atttccataattcattctcgccttcctatcttagattttcaatagttgtaagggttccatctagcacttaatctacaaaagtatctcactcaggcactga